(19) United States
(12) Patent Application Publication
(10) Pub. No.: US 2024/0036032 A1
(43) Pub. Date: Feb. 1, 2024

(54) PREDICTIVE IN VITRO ASSAY MIMICKING IN VIVO PHARMACOLOGY

(71) Applicant: MIMI-Q GMBH, Potsdam (DE)

(72) Inventors: Andrey POLOZNIKOV, Moscow (RU); Sergey NIKULIN, Ulyanovsk (RU)

(21) Appl. No.: 18/254,530

(22) PCT Filed: Dec. 1, 2021
(86) PCT No.: PCT/IB2021/061187

§ 371 Date: May 25, 2023

(30) Foreign Application Priority Data
Dec. 4, 2020 (SE) 2051418-8

(51) Int. Cl. G01N 33/50

(52) U.S. Cl. CPC G01N 33/5011

(57) ABSTRACT

A method is disclosed herein to reconstruct and mimic the in vivo pattern of exposure to the active substances included in the drug treatment regimen in vitro using a cell assay. Means and methods for converting an in vivo pattern of exposure to appropriate concentrations, durations of exposure and time points of addition for an in vitro assay.

### TECHNICAL FIELD

The present invention relates to the field of in vitro assays predicting the efficacy of a drug treatment regimen, particularly in the field of cancer therapy.

### BACKGROUND TO THE INVENTION

Despite recent progress in cancer diagnostics and treatment, cancer is still one of the top causes of death in the world. The major problem in cancer treatment is systemic spread of the disease in the body in the form of metastases. Metastases can form years after initial treatment or can be present at the diagnosis. In both cases current treatment regimens rarely achieve complete recovery of the patients with metastases and the treatment in this case is often palliative in nature. Therefore, new and improved methods to treat metastatic patients and to prevent the formation of the metastases are needed.

It is clear now that even one particular type of cancer is a heterogeneous group of diseases with different molecular and cellular nature and different optimal treatment strategies. Understanding of this fact led to the development and clinical implementation of various tests based on molecular characteristics of the tumor. These tests are especially useful for application of targeted cancer therapeutic drugs as these drugs has been designed to inhibit tumor cells harboring a particular molecular lesion. However, these tests do not guarantee efficiency of the chosen treatment course. For example, this is illustrated by low efficiency of anti-EGFR therapy. Increased expression or activation of the epidermal growth factor receptor (EGFR) is observed in 60-80% of colorectal cancer (CRC) cases [1], while only 10% of patients with chemo-resistant metastatic CRC respond to anti-EGFR therapy with cetuximab and panitumumab in the absence of genetic selection, and almost all patients who demonstrated an initial tumor response to targeted therapy in 3-12 months develop resistance to these drugs [1,2]. It was demonstrated that the initial resistance to anti-EGFR drugs can be caused by mutations in KRAS gene, however mutated KRAS was detected in only 68% of CRC tumors which did not respond to the therapy [3]. Despite the selection of patients based on the status of KRAS gene, only about 40% of them responded to the anti-EGFR therapy.

There are also several examples which show that personalized molecular tests can also be useful for appropriate application of chemotherapy. One of the most widely used such assays in clinic is OncotypeDX. This test allows to predict the risk of the relapse of early stage breast cancer. It has been shown that the patient with high risk of the relapse definitely benefit from standard chemotherapy used to treat breast cancer [4]. On the other hand, chemotherapy does not improve the survival of the patients with low or intermediate risk [5]. However, it is not still clear how to treat patients with intermediate or high risk to reach the survival similar to the low-risk group. All mentioned drawbacks of the molecular tests indicate the need to develop complimentary techniques to predict the efficacy of a treatment regimen for a particular patient.

In sharp contrast to wide implementation of molecular-based assays to direct the treatment of cancer patients currently there are no widely used cell-based assays which allow to predict the efficacy of a particular treatment regimen. The idea to test anti-cancer drugs on cancer cells like testing of antibiotics on bacterial cells appeared more than 50 years ago, however the tests did not enter clinical practice for several reasons. One of the most important was inability to establish cancer cell culture from sufficient number of patients. This problem has been solved recently and the methods of efficient generation of cancer cell cultures from various types of cancers have been published [6-8]. Despite utilization of these next-generation cancer cell models the correlation of the in vitro and clinical results can be insufficient sometimes meaning that advanced more precise methods of drug testing are needed [9].

Today there are a few widely used methods of the evaluation of the efficacy of anti-tumor agents and none of them accurately reflect the clinical situation and hardly can be used to predict the efficacy of currently used and prospective drug treatment regimens for each particular patient. One of the most popular method is calculation of half-inhibitory concentration (IC50) based on dose-response curve [10]. This method is appropriate for relative comparison of the efficiency of different drugs on one cell culture or susceptibility of different cells to one particular drug. It is possible to say based on the results of this type of the analysis which drug is more active or which cells are more resistant. However, this type of analysis are not accurately predictive of whether the drug will work in clinic or not. The reason for that is it has been designed for different purposes and the concentrations and durations of exposure used are often out of clinical range. Moreover, today most of clinically used regimens include sequential application of different agents and the IC50 method can measure the effect of only one drug.

Another approach which has also been described is so called “peak plasma concentration” or “one relevant concentration” method when a clinically relevant concentration (often maximal concentration in blood plasma) of a drug applied on cells [11]. In theory it is possible to mix several different drugs in such setting. However, this method does not take into account the sequential nature of drug application in clinical regimens [12]. Moreover, the concentrations and durations of exposure used are often too high and long leading to overtreatment of the cells and limited clinical relevance.

Thus, an object of the present invention is the provision of improved or at least alternative solution for testing of the impact of various treatment regimens on cancer cells.

### BRIEF DESCRIPTION OF THE FIGURES

FIG. 1. Schematic representation of the assay protocol for mFOLFOX6 regimen.

FIG. 2. Relative number of cells of colon cancer organoids after incubation with active substances of mFOLFOX6 protocol using two sets of parameters stated in Table 5.

FIG. 3. Relative number of cells of colon cancer organoids after incubation with active substances of mFOLFOX6 regimen using different methods. a—relative number of the colon cancer cells after incubation with active substances in accordance to criteria presented in Table 1 i.e. according to the present invention (FOLFOX method) and approach described in Romero-Calvo et al[11](FOLFOX Peak); b—relative number of the colon cancer cells after incubation with serial dilutions of 5-fluorouracil.

FIG. 4. Relative number of cells of colon cancer organoids after incubation with active substances of mFOLFOX6 and XELOX regimens using different methods. FOLFOX method and XELOX method—relative number of the colon cancer cells after incubation with active substances in accordance to the parameters presented in Table 6 i.e. according to the present invention; FOLFOX peak and XELOX peak—relative number of the colon cancer cells after incubation with active substances in accordance to approach described in Romero-Calvo et al[11].

FIG. 5. Relative number of cells of colon cancer organoids after incubation with active substances of FOLFIRI, XELOX and Capecitabine monotherapy regimens using the method in accordance to the parameters presented in Table 7 i.e. according to the present invention.

FIG. 6. Comparison of the clinical response and in vitro results using different methods for evaluating the efficacy of a drug treatment regimens. Clinical responders are presented as figures filled with white color, clinical non-responders are presented as figures filled with black color. In contrast to dose-response curves, the method of the invention provides correct prediction of clinical outcomes.

FIG. 7. Schematic representation of a device adapted for performing the method of the invention.

| Sign | Explanation |
|---|---|
| 1 | Sterile enclosure |
| 2 | Liquid handling unit (e.g. robotic pipetting station) |
| 3 | Microplate support with cooled areas |
| 4 | Storage unit (e.g. refrigerated reagent rack) |
| 5 | Microplate transport system |
| 6 | Detector for determining the phenotypical changes of tumor |
| | cells (e.g. microplate reader) |
| 7 | Programmable control unit (e.g. personal computer) |
| 8 | Incubator unit (e.g. CO₂ cell incubator) |

FIG. 8. Schematic flow chart of the predictive in vitro assay mimicking in vivo pharmacology according to the invention.

Stage A. Selecting a clinical drug treatment regimen to be evaluated.

Stage B. Identifying the sequence of drug administrations included in said drug treatment regimen.

Stage C. Identifying the time of drug administrations included in said drug treatment regimen.

Stage D. Identifying the active substances relevant for each identified administration.

Stage E. Selecting concentration(s) for incubation with each identified active substance corresponding to the in vivo concentration of said active substance typical for said drug treatment regimen.

Stage F. Selecting duration(s) for incubation with each identified active substance corresponding to the duration of clinically effective drug exposure to said active substance in said drug treatment regimen.

Stage G. Selecting time point(s) of addition for initiation of incubation with each identified active substance corresponding to the sequence and timing of drug administrations relevant for clinical exposure to said active substance in said drug treatment regimen.

Stage H. Providing a culture of tumor cells for an in vitro assay.

Stage I. Culturing the tumor cells in vitro with addition of each identified active substance(s) at the selected time point(s) of addition in accordance with the sequence and incubating in the presence of the selected concentration(s) of said active substance(s) for the selected duration(s).

Stage J. Determining the phenotypical changes of the tumor cells due to effect of the active substance(s).

Stage K. Evaluating the efficacy of the drug treatment regimen based on the observed phenotypical changes.

FIG. 9. Generic examples of the various concentration-time curves.

A. Concentration-time curve of drug that is rapidly metabolized and excreted and administered by bolus injection.

B. Concentration-time curve of drug that is rapidly metabolized and excreted and administered by infusion.

C. Concentration-time curve of drug that is slowly metabolized and excreted and administered by bolus injection.

FIG. 10. Schematic representation of possible reference clinical parameters.

A. Maximum concentration observed in the body Cmax.

B. Area under concentration-time curve.

FIG. 11. Schematic representation of translation of clinical parameters of a drug to parameters of assay.

A. Generic example of the clinical concentration-time curve of drug that is rapidly metabolized and excreted and administered by bolus injection.

B. Generic example of the clinical concentration-time curve of drug that is slowly metabolized and excreted and administered by bolus injection.

C. The concentration-time curve obtained via translation of clinical parameter of generic example A into assay parameters of drug that is rapidly metabolized and excreted and administered by bolus injection.

D. The concentration-time curve obtained via translation of clinical parameter of generic example B into assay parameters of drug that is slowly metabolized and excreted and administered by bolus injection.

FIG. 12. Schematic representation of possible reference clinical parameters for evaluation of a time of presence of an active substance in the body.

A. Generic example of the concentration-time curve of the drug that is administered by bolus injection, orally or any other type excluding infusion.

B. Generic example of the concentration-time curve of the drug that is administered by infusion.

FIG. 13. Schematic representation of evaluation of simultaneous presence of active substances in the body and translation the result of evaluation to the parameters of assay.

A. Generic example of the clinical concentration-time curves of drugs that are considered to be present simultaneously in the body.

B. Generic example of the clinical concentration-time curves of drugs that are considered not to be present simultaneously in the body.

C. The concentration-time curves obtained via translation the result of evaluation of simultaneous presence of active substances in the body of generic example A into assay parameters.

D. The concentration-time curves obtained via translation the result of evaluation of simultaneous presence of active substances in the body of generic example C into assay parameters.

### SUMMARY OF THE INVENTION

In order to recapitulate the in vivo response to a particular drug treatment regimen in vitro, a method is disclosed herein to reconstruct and mimic the in vivo pattern of exposure to the active substances included in the drug regimen in vitro.

Usually a drug treatment regimen, particularly a cancer treatment regimen, consists of introduction of multiple drugs (at least one), at multiple time points (at least one) and by different routes (at least one). The combination of multiple drugs and manners of administration lead to a characteristic concentration-time profile of the active substances which the targeted cells are exposed to in the drug treatment regimen.

According to the invention, these characteristic concentration-time profiles can be reconstructed in vitro with sufficient fidelity to improve clinical relevance for the test compared to traditional dose-response testing.

The present invention provides methods for transforming known or predicted in vivo pharmacokinetics profiles of the drugs included in a drug treatment regimen into sufficiently similar in vitro profiles which can be recapitulated in a lab. The methods allow testing of regimens which are already used in clinic as well as experimental regimens with different dosing and scheduling or including new drug candidates. The methods can be used to create in vitro concentration-time profiles suitable for manually performed experiments or suitable for handling by automated robotic systems.

Example 1 illustrates conversion of mFOLFOX6 drug regiment to a useful in vitro profile. In Example 2, the inventors show proof-of-concept of the analysis using patient material. As shown in Example 3, the method of the invention provided more precise results than previous methods used for evaluation of the efficacy of a drug treatment regimen.

In Example 4 the inventors demonstrate that clinically similar treatment regimens also resulted in similar results in an in vitro assay performed in accordance with the invention.

Example 5 provides proof-of-concept that the method of the invention can be used to select a beneficial drug treatment regimen for a patient in need thereof.

The present invention relates to the following items. The subject matter disclosed in the items below should be regarded disclosed in the same manner as if the subject matter were disclosed in patent claims.

1. An in vitro method for evaluating the efficacy of a clinical drug treatment regimen utilizing tumor cells, comprising the steps of:
a. selecting a clinical drug treatment regimen to be evaluated comprising more than one drug and/or more than one instances of administering a drug, noting the sequence and timing of drug administrations included in said drug treatment regimen and identifying the one or more active substance(s) relevant for each noted administration;
b. selecting in vitro culture parameters corresponding to each noted drug administration comprising:
   i. selecting concentration(s) for incubation with each identified active substance corresponding to the in vivo concentration of said active substance typical for said drug treatment regimen;
   ii. selecting duration(s) of exposure for incubation with each identified active substance corresponding to the duration of clinically effective drug exposure to said active substance in said drug treatment regimen; and
   iii. selecting time point(s) of addition for initiation of incubation with each identified active substance corresponding to the sequence and timing of drug administrations relevant for clinical exposure to said active substance in said drug treatment regimen; wherein the combination of the selected time point(s), duration(s) of exposure and concentrations(s) imitates the clinical exposure profile typical for said drug treatment regimen;
c. providing a culture of tumor cells for an in vitro assay;
d. culturing the tumor cells in vitro with addition of each identified active substance(s) at the selected time point(s) of addition in accordance with the sequence and incubating in the presence of the selected concentration(s) of said active substance(s) for the selected duration(s);
e. determining the phenotypical changes of the tumor cells due to effect of the active substance(s); and
f. evaluating the efficacy of the drug treatment regimen based on the observed phenotypical changes.

2. The method according to item 1, wherein:
a. the method comprises identification of the active substance(s) of said drug treatment regimen that are simultaneously present in the body based on the sequence and timing of drug administrations and pharmacokinetic parameters of said active substance(s) such as pharmacological half-life and in vivo time for the active substance to reach its maximum; and
b. wherein the selected time point(s) of addition are for sequential initiation of incubation, based on the simultaneous presence in the body.

3. The method according to any of the preceding items, wherein the method comprises selecting a total in vitro assay time Tₜₒₜ, and wherein for each identified active substance, the duration Tₑₓ of exposure for in vitro assay and in vitro concentration C_{in vitro} for assay, are selected to satisfy the following rules:

$$i . 0.01 \cdot {C}_{max in vivo} \leq {C}_{in vitro} \leq 3 \cdot {C}_{max in vitro} ;$$

$$ii . \frac{0.2 \cdot {AUC}_{in vivo}}{{C}_{in vitro}} \leq {T}_{ex} \leq \frac{3 \cdot {AUC}_{in vivo}}{{C}_{in vitro}} ;$$

where the in vivo maximal concentration C_{max in vivo} for each active substance and the in vivo area under concentration-time curve AUC_{in vivo} for the total in vitro assay time Tₜₒₜ for each active substance are based on clinical data for the selected regimen.

4. The method according to any of the preceding items, wherein the selected sequence and time point(s) of addition match the sequence of administrations in the selected regimen within a margin of ±8 hours, preferably ±4 hours, more preferably ±2 hours, most preferably ±1 hours.

5. The method according to any of the preceding items, wherein the drug treatment regimen is selected from the list consisting of FOLFOX, FOLFIRI, XELOX and capecitabine monotherapy.

6. The method according to any of the preceding items, wherein any active substances which do not possess direct cytotoxic or cytostatic effect are ignored.

7. The method according to any of the preceding items, wherein an active substance is the drug itself, an active drug metabolite or the drug or its metabolite in complex with other compound(s).

8. The method according to any of the preceding items, wherein culturing the tumor cells is performed in 2D cell culture, suspension cell culture or 3D cell culture including tumor organoids or their combination with stromal and immune cells.

9. The method according to any of the preceding items, wherein the cells are cell lines or primary cells.

10. The method according to any of the preceding items, wherein the drug treatment regimen is a chemotherapy, targeted therapy, immunotherapy treatment regimen for cancer or their combinations.

11. The method according to any of the preceding items, wherein the tumor cells are derived from an individual patient afflicted with a tumor disease, and the method is performed to evaluate the efficacy of a chemotherapy, targeted therapy, immunotherapy or their combinational drug treatment regimen in the treatment of said tumor disease.

12. The method according to any of the preceding items, wherein drug administration(s) included in the drug treatment regimen are delivered to the body by infusion, bolus injection or orally.

13. The method according to any of the preceding items, wherein the time point of addition for the first substance in a sequence is considered as a zero-time point of the entire sequence.

14. The method according to any of the preceding items, wherein the drug treatment regimen comprises more than two drugs and/or more than two instances of administering a drug.

15. The method according to any of the preceding items, wherein the selected in vitro culture parameters comprise at least two time points of addition and/or at least two durations of exposure.

16. The method according to any of the preceding items, wherein the selected in vitro culture parameters comprise active substance concentration(s) that vary over time during the tumor cell culture.

17. The method according to any of the preceding items, wherein a plurality of combinations of the concentration and duration of exposure are selected for each identified substance.

18. The method according to any of the preceding items, wherein the phenotypical change is a change in the number of cells.

19. The method according to any of the preceding items, wherein the phenotypical change is cell proliferation or cell death.

20. The method according to any of the preceding items, wherein the drug treatment regimen is considered effective when this regimen inhibits cell proliferation, stops cell proliferation and/or causes cell death.

21. The method according to any of the preceding items, wherein the selected concentration(s) satisfy the conditions as defined in Table 1.

22. The method according to any of the preceding items, wherein the selected duration(s) satisfy the conditions as defined in Table 1.

23. The method according to any of the preceding items, wherein the selected time point(s) of addition satisfy the conditions as defined in Table 1.

24. The method according to any of the preceding items, wherein the selecting in vitro culture parameters is performed in accordance with the method of item 25 or any item dependent thereon.

25. A method for selecting in vitro culture parameters for a method according to item 1 or any item dependent thereon, comprising the steps of:
a. selecting a drug treatment regimen to be evaluated;
b. selecting a total in vitro assay time Tₜₒₜ;
c. identifying the sequence [1, . . . , l] and time T_{a in vivo}ⁱ ([T_{a in vivo}¹, . . . , T_{a in vivo}^{l}]) of drug administrations included in said drug treatment regimen to be evaluated during Tₜₒₜ;
d. identifying the active substances Sₖⁱ ([S₁ⁱ, . . . , S_{K}ⁱ]) relevant for each identified administration i;
e. identifying clinical half life time T_{1/2[k]}ⁱ ([T_{1/2[1]}ⁱ, . . . , T_{1/2[k]}ⁱ]) for each active substance Sₖⁱ for each administration i;
f. identifying clinical time T_{max[k]}ⁱ ([T_{max[1]}ⁱ, . . . , T_{max[K]}ⁱ]) for each active substance Sₖⁱ when concentration of this active substance reaches its maximum;
g. identifying clinical time of presence T_{pr[k]}ⁱ ([T_{pr[1]}ⁱ, . . . , T_{max[K]}^{K}]) for each active substance Sₖⁱ when this active substance is present in plasma in the range between T_{max[k]}ⁱ+0.1·T_{1/2[k]}ⁱ and T_{max[k]}ⁱ+10·T_{1/2[k]}ⁱ;
h. determining whether there is simultaneous presence of each identified active substance Sₖⁱ and any of the active substance(s) S_{y}^{x} of the preceding administration(s) and current administration i using the following rules:
   i. if T_{a in vivo}ⁱ−T_{a in vivo}^{x}≥T_{pr[y]}^{x} then the substances are considered not to be present simultaneously in the body;
   ii. if T_{a in vivo}ⁱ−T_{a in vivo}^{x}<T_{pr[y]}^{x} then the substances are considered to be present simultaneously in the body;
i. identifying the in vivo area under the curve AUC_{in vivo[k]}ⁱ for the period Tₜₒₜ for each active substance Sₖⁱ for each administration i, based on clinical data for the selected regimen;
j. identifying the in vivo maximal concentration C_{max in vivo[k]}ⁱ for each active substance Sₖⁱ for each administration i, based on clinical data for the selected regimen;
k. for each identified active substance Sₖⁱ, selecting at least one combination (when applicable: several combinations 1, . . . , N) of duration T_{ex[k][n]}ⁱ of exposure for in vitro assay and in vitro concentration C_{in vitro [k][n]}ⁱ for assay, to satisfy the following rules:

$$i . 0.01 \cdot {C}_{max in vivo [ k ]}^{i} \leq {C}_{in vitro [ k ] [ 1 ]}^{i} \leq 3 \cdot {C}_{max in vivo [ k ]}^{i} ;$$

$$ii . \frac{0.2 \cdot {AUC}_{in vivo [ k ]}^{i}}{{C}_{in vitro [ k ] [ 1 ]}^{i}} \leq {T}_{ex [ k ] [ 1 ]}^{i} \leq \frac{3 \cdot {AUC}_{in vivo [ k ]}^{i}}{{C}_{in vitro [ k ] [ 1 ]}^{i}} ;$$

l. optionally: for each identified active substance Sₖⁱ, if C_{in vitro[k][1]}ⁱ·T_{ex[k][1]}ⁱ<3·AUC_{in vivo[k]}ⁱ then additional combinations of duration T_{ex[k][n]}ⁱ of exposure for assay and concentration C_{in vitro [k][n]}ⁱ for assay are identified to satisfy the following rules:

0.01·C_{max in vivo[k]}ⁱ≤C_{in vitro [k][n]}ⁱ≤3·C_{max in vivo[k]}ⁱ; i.

0.2·AUC_{in vivo[k]}ⁱ≤AUC_{in vitro[k]}ⁱ≤3·AUC_{in vivo[k]}ⁱ, where AUC_{in vitro[k]}ⁱ=AUC_{in vitro[k][1]}ⁱ+ . . . +AUC_{in vitro[k][N]}ⁱ and AUC_{in vitro[k][n]}ⁱ=C_{in vitro [k][n]}ⁱ·T_{ex[k][n]}ⁱ; ii.
m. for all identified administrations and all identified active substances sum of all durations of exposure for assay must satisfy the following criterion:

ΣT_{ex[k][n]}ⁱ≤Tₜₒₜ;
n. for each identified active substance Sₖⁱ relevant for the first identified administration time point of addition in vitro T_{a in vitro[k]}¹ for assay must satisfy the following rule: 0≤T_{a in vitro[k]}¹≤T_{max[k]}¹; and
o. for each identified active substance Sₖⁱ relevant for any administration except the first one selecting time point of addition in vitro T_{a in vitro[k]}ⁱ for assay must satisfy the following rules:
   i. if identified active substance Sₖⁱ and any of the active substance(s) S_{y}^{x} of the preceding administration(s) and current administration l are present simultaneously in the body then T_{a in vitro[y]}^{x}≤T_{a in vitro[k]}ⁱ≤T_{a in vitro[y]}^{x}+ΣₙT_{ex[y][n]}^{x};
   ii. if identified active substance Sₖⁱ and any of the active substance(s) S_{y}^{x} the preceding administration(s) and current administration l are not present simultaneously in the body then T_{a in vitro[y]}^{x}+ΣₙT_{ex[y][n]}^{x}≤T_{a in vitro[k]}ⁱ<Tₜₒₜ.

26. The method according to item 25, wherein Tₜₒₜ is the time needed for the tumor cells to increase by at least 1.2-fold in cell numbers in culture.

27. The method according to any of items 25-26, wherein Tₜₒₜ≤72 h.

28. The method according to any of the preceding items, wherein the selected drug treatment regimen is FOLFOX, and the selected in vitro culture parameters satisfy the following criteria:

| | | Substance | Duration | Time point |
|---|---|---|---|---|
| Sequence | | concentration | of exposure | of addition |
| number | Substance | range (μM) | range (h) | range (h) |
| 1 | Oxaliplatin | 0.2-10.8 | 0.13-24 | 0-0 |
| 2 | 5-fluorouracil | 15-400 | 0.03-5 | 0.13-24 |
| 3 | 5-fluorouracil | 0.45-16.5 | 2-72 | 0.13-29 |

29. The method according to any of the preceding items, wherein the selected drug treatment regimen is XELOX, and the selected in vitro culture parameters satisfy the following criteria:

| | | Substance | Duration | Time point |
|---|---|---|---|---|
| Sequence | | concentration | of exposure | of addition |
| number | Substance | range (μM | range (h) | range (h) |
| 1 | Oxaliplatin | 0.5-41 | 0.13-24 | 0-0 |
| 2 | 5-fluorouracil | 0.5-41 | 0.15-12 | 0.13-36 |
| 3 | 5-fluorouracil | 0.5-41 | 0.15-12 | 0.28-48 |
| n | 5-fluorouracil | 0.5-41 | 0.15-12 | 0.13 + |
| | | | | (n − 2)*0.15 |
| | | | | to 36 + 12* |
| | | | | (n − 2) |

30. The method according to any of the preceding items, wherein the selected drug treatment regimen is FOLFIRI, and the selected in vitro culture parameters satisfy the following criteria:

| | | Substance | Duration | Time point |
|---|---|---|---|---|
| Sequence | | concentration | of exposure | of addition |
| number | Substance | range (μM | range (h) | range (h) |
| 1 | CPT-11 | 0.1-10.89 | 0.35-72 | 0-0 |
| 1 | SN-38 | 0.002-0.266 | 0.35-72 | 0-2.3 |
| 2 | 5-fluorouracil | 15-400 | 0.03-5 | 0.35-72 |
| 3 | 5-fluorouracil | 0.45-16.5 | 2-72 | 0.35-72 |

31. The method according to any of the preceding items, wherein the selected drug treatment regimen is capecitabine monotherapy, and the selected in vitro culture parameters satisfy the following criteria:

| | | Substance | Duration | Time point |
|---|---|---|---|---|
| Sequence | | concentration | of exposure | of addition |
| number | Substance | range (μM | range (h) | range (h) |
| 1 | 5-fluorouracil | 0.5-41 | 0.15-12 | 0-0 |
| 2 | 5-fluorouracil | 0.5-41 | 0.15-12 | 0.15-24 |
| 3 | 5-fluorouracil | 0.5-41 | 0.15-12 | 0.3-36 |
| n | 5-fluorouracil | 0.5-41 | 0.15-12 | 0.15 + |
| | | | | (n − 2)*0.15 |
| | | | | to 24 + 12* |
| | | | | (n − 2) |

32. A device adapted to perform the method of any of the preceding items, comprising:
a. a programmable control unit (7) for executing device functions to perform the method;
b. an incubator unit (8) for in vitro tumor cell culture;
c. a storage unit (4) for the active compound(s);
d. a liquid handling unit (2) for administering the active compound(s) to cultured tumor cells in accordance with the method; and
e. a detector for determining the phenotypical changes of tumor cells (6) due to effect of the active substance(s).

33. A computer program product comprising instructions to cause the device of item 32 to execute the steps of the method of item 1 or any item dependent thereon.

34. A computer-readable data carrier having stored thereon the computer program product of item 33.

35. A computer-implemented method according to item 25 or any item dependent thereon.

36. A data processing device comprising means for carrying out the steps of the method of item 35.

37. A computer program product comprising instructions which, when the program is executed by a computer, cause the computer to carry out the steps of the method of item 35.

38. A computer-readable data carrier having stored thereon the computer program product of item 37.

39. A data carrier signal carrying the computer program product of item 37.

40. A method treatment for cancer in a patient in need thereof, comprising performing an analysis in accordance with the method of item 1 or any item dependent thereon using cells derived from the patient, whereby various drug treatment regimen are tested using the cells, and based on the results obtained, said patient is subsequently administered with a drug treatment regimen deemed most likely to be beneficial for said patient to treat the cancer.

### DETAILED DESCRIPTION

### In Vitro Evaluation of Drug Treatment Efficacy

In a first aspect, the present invention provides an in vitro method for evaluating the efficacy of a clinical drug treatment regimen utilizing tumor cells, comprising the steps of:
a. selecting a clinical drug treatment regimen to be evaluated comprising more than one drug and/or more than one instances of administering a drug, noting the sequence and timing of drug administrations included in said drug treatment regimen and identifying the one or more active substance(s) relevant for each noted administration;
b. selecting in vitro culture parameters corresponding to each noted drug administration comprising:
   i. selecting concentration(s) for incubation with each identified active substance corresponding to the in vivo concentration of said active substance typical for said drug treatment regimen;
   ii. selecting duration(s) for incubation with each identified active substance corresponding to the duration of clinically effective drug exposure to said active substance in said drug treatment regimen; and
   iii. selecting time point(s) of addition for initiation of incubation with each identified active substance corresponding to the sequence and timing of drug administrations relevant for clinical exposure to said active substance in said drug treatment regimen; wherein the combination of the selected time point(s), duration(s) and concentrations(s) imitates the clinical exposure profile typical for said drug treatment regimen;
c. providing a culture of tumor cells for an in vitro assay;
d. culturing the tumor cells in vitro with addition of each identified active substance(s) at the selected time point(s) of addition in accordance with the sequence and incubating in the presence of the selected concentration(s) of said active substance(s) for the selected duration(s);
e. determining the phenotypical changes of the tumor cells due to effect of the active substance(s); and
f. evaluating the efficacy of the drug treatment regimen based on the observed phenotypical changes.

The drug treatment regimen can be chosen from clinically used cancer treatment regimens or can be an experimental one. The drug treatment regimen may be a chemotherapy, targeted therapy, immunotherapy treatment regimen for cancer or their combinations. The drug treatment regimen can include one drug or can include several drugs. The included drugs can be approved for clinical use ones or experimental ones. The included drugs can be administered multiple times or one time within the treatment regimen.

Preferably, the drug treatment regimen comprises more than two drugs and/or more than two instances of administering a drug, more preferably more than three drugs and/or more than three instances of administering a drug, even more preferably more than four drugs and/or more than four instances of administering a drug, most preferably more than five drugs and/or more than five instances of administering a drug.

The method may preferably comprise identification of the active substance(s) of said drug treatment regimen that are simultaneously present in the body based on the sequence and timing of drug administrations and pharmacokinetic parameters of said active substance(s) such as pharmacological half-life and in vivo time for the active substance to reach its maximum, wherein the selected time point(s) of addition are for sequential initiation of incubation, based on the simultaneous presence in the body.

The method may comprise selecting a total in vitro assay time Tₜₒₜ, and wherein for each identified active substance, the duration Tₑₓ of exposure for in vitro assay and in vitro concentration C_{in vitro} for assay, are selected to satisfy the following rules:

$$i . 0.01 \cdot {C}_{max in vivo} \leq {C}_{in vitro} \leq 3 \cdot {C}_{max in vivo} ;$$

$$ii . \frac{0.2 \cdot {AUC}_{in vivo}}{{C}_{in vitro}} \leq {T}_{ex} \leq \frac{3 \cdot {AUC}_{in vivo}}{{C}_{in vitro}} ;$$

where the in vivo maximal concentration C_{max in vivo} for each active substance and the in vivo area under the curve AUC_{in vivo} for the total in vitro assay time Tₜₒₜ for each active substance are based on clinical data for the selected regimen. The Tₜₒₜ may be the time needed for the tumor cells to increase by at least 1.2-fold in cell numbers in culture. Preferably, Tₜₒₜ≤72 h.

The selected sequence and time point(s) of addition may match the sequence of administrations in the selected regimen within a margin of ±8 hours, preferably ±4 hours, more preferably ±2 hours, most preferably ±1 hours.

Preferably, the selected in vitro culture parameters comprise at least two time points of addition and/or at least two durations of exposure, more preferably at least three time points of addition and/or at least three durations of exposure, even more preferably at least four time points of addition and/or at least four durations of exposure, most preferably at least five time points of addition and/or at least five durations of exposure.

Non-limiting list of the drugs used as cancer monotherapy or in combination with other drugs is provided on the website: cancer.gov/about-cancer/treatment/drugs. The list also contains several combinational cancer drug treatment regimens which include several drugs. Thus, the cancer drug treatment regimens may be selected from the list consisting of monotherapy treatment regimens or combinational treatment regimens, preferably combinatorial regimens:

Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPDX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fam-Trastuzumab Deruxtecan-nxki, Fareston (Toremifene), Farydak (Panobinostat), Faslodex (Fulvestrant), FEC, Fedratinib Hydrochloride, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Infugem (Gemcitabine Hydrochloride), Inlyta (Axitinib), Inotuzumab Ozogamicin, Inciovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), lobenguane I 131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, lxempra (Ixabepilone), Jakafi (Ruxolitinib Phosphate), JEB, Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lomustine, Lonsurf (Trifluridine and Tipiracil Hydrochloride), Lorbrena (Lorlatinib), Lorlatinib, Lumoxiti (Moxetumomab Pasudotox-tdfk), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin, Mitoxantrone Hydrochloride, Mogamulizumab-kpkc, Monjuvi (Tafasitamab-cxix), Moxetumomab Pasudotox-tdfk, Mozobil (Plerixafor), MVAC, Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Pa pillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoetin Alfa), Retevmo (Selpercatinib), Revlimid (Lenalidomide), Rheumatrex (Methotrexate), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Trastuzumab and Hyaluronidase-oysk, Treanda (Bendamustine Hydrochloride), Trexall (Methotrexate), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), Zytiga (Abiraterone Acetate).

In one aspect, the present invention also provides a method treatment for cancer in a patient in need thereof, comprising performing an analysis in accordance with the method of the first aspect using cells (preferably tumor cells) derived from the patient, whereby various drug treatment regimen are tested using the cells, and based on the results obtained, said patient is subsequently administered with a drug treatment regimen deemed most likely to be beneficial for said patient to treat the cancer.

Thus, the drug treatment regimen may be selected, for example, from the list consisting of FOLFOX, FOLFIRI, XELOX and capecitabine monotherapy. The regimen FOLFOX, includes administration(s) of the following drugs: oxaliplatin, 5-fluorouracil (5-FU) and Leucovorin Calcium (Folinic Acid). The regimen FOLFIRI, includes administration(s) of the following drugs: Irinotecan (CPT-11), 5-fluorouracil (5-FU) and Leucovorin Calcium (Folinic Acid). The regimen XELOX, includes administration(s) of the following drugs: oxaliplatin and capecitabine. The regimen capecitabine monotherapy, includes administration(s) of capecitabine.

Any drug treatment regimen consists of at least one administration of at least one drug. For example, one version of the regimen FOLFOX-FOLFOX4 consists of 2 administrations of oxaliplatin, 4 administrations of 5-fluorouracil (5-FU) and 2 administrations of Leucovorin Calcium (Folinic Acid). For example, another version of the regimen FOLFOX-FOLFOX6 consists of 1 administration of oxaliplatin, 2 administrations of 5-fluorouracil (5-FU) and 1 administration of Leucovorin Calcium (Folinic Acid). For example, the regimen FOLFIRI consists of 1 administration of Irinotecan (CPT-11), 2 administrations of 5-fluorouracil (5-FU) and 1 administration of Leucovorin Calcium (Folinic Acid). For example, the regimen XELOX consists of 1 administration of oxaliplatin, 28 administrations of Capecitabine. For example, the regimen XELOX consists of 1 administration of oxaliplatin, 28 administrations of Capecitabine. For example, the regimen capecitabine monotherapy consists of 28 administrations of Capecitabine.

The administrations may be performed via different methods. Non-limiting list of possible methods consists of intravenous infusion, bolus injection, oral administration and intramuscular injection. For example, in one version of the regimen FOLFOX-FOLFOX4 2 administrations of oxaliplatin are performed by intravenous infusion, 2 administrations of 5-fluorouracil (5-FU) are performed by bolus injection, 2 administrations of 5-fluorouracil (5-FU) are performed by intravenous infusion and 2 administrations of Leucovorin Calcium (Folinic Acid) are performed by intravenous infusion. For example, in another version of the regimen FOLFOX-FOLFOX6 1 administration of oxaliplatin is performed by intravenous infusion, 1 administration of 5-fluorouracil (5-FU) is performed by bolus injection, 1 administration of 5-fluorouracil (5-FU) is performed by intravenous infusion and 1 administration of Leucovorin Calcium (Folinic Acid) is performed by intravenous infusion. For example, in the regimen FOLFIRI 1 administration of Irinotecan (CPT-11) is performed by intravenous infusion, 1 administration of 5-fluorouracil (5-FU) is performed by bolus injection, 1 administration of 5-fluorouracil (5-FU) is performed by intravenous infusion and 1 administration of Leucovorin Calcium (Folinic Acid) is performed by intravenous infusion. For example, in the regimen XELOX 1 administration of oxaliplatin is performed by intravenous infusion and 28 administrations of Capecitabine are performed orally. For example, in the regimen capecitabine monotherapy 28 administrations of Capecitabine are performed orally.

The administrations are performed at defined sequence. For example, in one version of the regimen FOLFOX-FOLFOX4 1 intravenous infusion of oxaliplatin is performed concurrently with 1 intravenous infusion of Leucovorin Calcium, these infusions are followed by 1 bolus injection of 5-fluorouracil (5-FU) which is followed by 1 intravenous infusion of 5-fluorouracil (5-FU) and then 1 intravenous infusion of oxaliplatin is performed concurrently with 1 intravenous infusion of Leucovorin Calcium, these infusions are followed by 1 bolus injection of 5-fluorouracil (5-FU) which is followed by 1 intravenous infusion of 5-fluorouracil (5-FU). For example, in another version of the regimen FOLFOX-FOLFOX6 1 intravenous infusion of oxaliplatin is performed concurrently with 1 intravenous infusion of Leucovorin Calcium, these infusions are followed by 1 bolus injection of 5-fluorouracil (5-FU) which is followed by 1 intravenous infusion of 5-fluorouracil (5-FU). For example, in the regimen FOLFIRI 1 intravenous infusion of Irinotecan (CPT-11) is performed concurrently with 1 intravenous infusion of Leucovorin Calcium, these infusions are followed by 1 bolus injection of 5-fluorouracil (5-FU) which is followed by 1 intravenous infusion of 5-fluorouracil (5-FU). For example, in the regimen XELOX 1 intravenous infusion of oxaliplatin is followed by 28 sequential oral administrations of Capecitabine. For example, in the regimen capecitabine monotherapy there are 28 sequential oral administrations of Capecitabine.

The administrations are performed with defined timings. Each administration may start at defined time point or there may be preferable time ranges of start points for a particular administration. Each administration may have a duration or it can be instant. For example, in one version of the regimen FOLFOX-FOLFOX4 the first intravenous infusion of oxaliplatin and the first intravenous infusion of Leucovorin Calcium are started simultaneously at zero time point and last for 2 hours, then after 2 hours the first bolus injection of 5-fluorouracil (5-FU) is performed (bolus injection usually lasts about 2 minutes, however it can be considered to be instant) which is immediately followed by the first intravenous infusion of 5-fluorouracil (5-FU) which lasts 22 hours and then at the time point of 24 hours from the beginning the second intravenous infusion of oxaliplatin and the second intravenous infusion of Leucovorin Calcium are started simultaneously and last for 2 hours, then after 2 hours at the time point of 26 hours the second bolus injection of 5-fluorouracil (5-FU) is performed (bolus injection usually lasts about 2 minutes, however it can be considered to be instant) which is immediately followed by the second intravenous infusion of 5-fluorouracil (5-FU) which lasts 22 hours. For example, in another version of the regimen FOLFOX-FOLFOX6 the intravenous infusion of oxaliplatin and the intravenous infusion of Leucovorin Calcium are started simultaneously at zero time point and last for 2 hours, then after 2 hours the bolus injection of 5-fluorouracil (5-FU) is performed (bolus injection usually lasts about 2 minutes, however it can be considered to be instant) which is immediately followed by the intravenous infusion of 5-fluorouracil (5-FU) which lasts 46 hours. For example, in the regimen FOLFIRI the intravenous infusion of Irinotecan (CPT-11) and the intravenous infusion of Leucovorin Calcium are started simultaneously at zero time point and the infusion of Irinotecan (CPT-11) lasts for 1.5 hours and the infusion of Leucovorin Calcium lasts for 2 hours, then after 2 hours the bolus injection of 5-fluorouracil (5-FU) is performed (bolus injection usually lasts about 2 minutes, however it can be considered to be instant) which is immediately followed by the intravenous infusion of 5-fluorouracil (5-FU) which lasts 46 hours. For example, in the regimen XELOX the intravenous infusion of oxaliplatin is started at zero time point and lasts for 2 hours, then in the evening of the same day (usually within 12 hours from the starting point) the first oral administration of capecitabine is performed (oral administration usually lasts less than 1 minute and can be considered to be instant) and then oral administrations of capecitabine are performed twice daily in the morning and in the evening. The last oral administration of capecitabine is performed on the fifteenth day in the morning. As in the regimen XELOX there are no strict indications on when oral administration should be performed it can be considered that capecitabine is administered for the first time in 12 hours from the start of the treatment and each subsequent administration is performed in 12 hours. For example, in the regimen capecitabine monotherapy oral administration of capecitabine (oral administration usually lasts less than 1 minute and can be considered to be instant) is performed twice daily in the morning and in the evening. As in the regimen capecitabine monotherapy there are no strict indications on when oral administration should be performed it can be considered that capecitabine is administered for the first time at zero time point and each subsequent administration is performed in 12 hours.

Any drug administration will include at least one active substance. An active substance may be the drug itself, an active drug metabolite or the drug or its metabolite in complex with other compound(s). For example, 5-fluorouracil (5-FU) possess antitumor activity itself and there are no other major known active substances which are associated with administration of 5-fluorouracil (5-FU). Thus, just one active substance (5-FU) may be taken into account for administrations of 5-fluorouracil (5-FU). Another example is oxaliplatin which is active by itself and has several inactive metabolites. Thus, just one active substance (oxaliplatin) may be taken into account for administrations of oxaliplatin. Leucovorin Calcium is an adjunct drug that has no direct antitumor activity. Any active substances which do not possess direct cytotoxic or cytostatic effect need not be considered in the in vitro method for evaluating the efficacy of a clinical drug treatment regimen utilizing tumor cells. Thus, no active substances are taken into account for administrations of Leucovorin Calcium. In general terms, any active substances which do not possess direct cytotoxic or cytostatic effect are optional for the analysis i.e. may be ignored. Irinotecan (CPT-11) has one major active metabolite SN-38. In addition, there have been published the data supporting the idea that Irinotecan (CPT-11) possess its own antitumor activity and it may also be considered to be an active substance. Thus, 2 active substances (CPT-11 and SN-38) may be taken into account for administrations of Irinotecan (CPT-11). It is known that active metabolite of capecitabine is 5-fluorouracil (5-FU), so just one active substance (5-FU) needs to be taken into account for administrations of capecitabine.

After a drug is administered it is absorbed into the bloodstream, distributed to the body's tissues, metabolized and excreted. Therefore, the active substance concentration is variable over the time. The profile of concentration-time curve can be determined by several parameters including but not limited to administration method and nature of the drug. For example, if a drug is rapidly metabolized and excreted e.g. 5-fluorouracil (5-FU) and administered by bolus injection, then the concentration profile will have a form of a sharp peak (FIG. 9A). In contrast if the same drug is administered by infusion, the concentration will be stable during the infusion period (FIG. 9B) and after that it will rapidly decrease. On the other hand, if the drug is slowly metabolized and excreted e.g. Bevacizumab, then the concentration of the drug will slowly decrease during a long period of time even after the end of the administration (FIG. 9C). Therefore, it is important to consider the clinical pharmacokinetics parameter of each drug for the determination of in vitro parameters for the evaluation of the efficacy of anti-tumor agents.

The selected in vitro culture parameters may comprise active substance concentration(s) that vary over time during the tumor cell culture.

To avoid overtreatment and undertreatment of cells in the in vitro assay it necessary to identify appropriate concentration and duration of exposure for each active substance. One of the possible references for identification of in vitro active substance concentration is its maximum concentration observed in the body Cₘₐₓ. For example, it can be maximum plasma concentration (FIG. 10A). Another important parameter is the area under concentration-time curve (AUC) which depends on the drug concentration and duration of its exposure (FIG. 10B). The in vitro active substance concentration C_{in vitro} and duration of exposure Tₑₓ parameters should be chosen in a way that resulting in vitro area under curve parameter is close enough to the clinical area under curve. As the present invention describes the drug treatment regimens comprising more than one drug and/or more than one instances of administering a drug, these parameters along with time point of addition that is described below should be chosen in a way that substances of different administrations are not mixed together in vitro if they are not present simultaneously in the body. In contrast, the substances should be mixed in vitro for a certain period of time if they are present simultaneously in the body. If after administration of a drug, its active substances are present in a body for a certain period of time during which the next drug is not administered, then there should be a similar period of time in vitro when the active substances of the drug are not present together with the active substances of the next administered drug. Therefore, it is important to sequentially add active substances in vitro to imitate their composition in the body at certain time points.

In some cases, for each administration of a particular drug of the chosen treatment regimen even one combination of in vitro active substance concentration and duration of exposure parameters is enough to recapitulate the in vivo concentration-time curve for the administration (FIGS. 11A, C). However, the present invention does not describe testing a single drug in vitro with one concentration and duration. As there are another administration(s) of other drugs (or the same drug) an additional parameter, time point of addition of each active substance, should be taken into account, and it depends on the fact whether active substances of different administrations and/or different drugs are present simultaneously in the body. Moreover, even if drugs are added at the same time, each of them should have individual duration of exposure. Therefore the assay contains multiple independent incubations of the identified active substances.

In other cases when an active substance is present in the body for a long period of time and wherein its concentration is significantly variable plurality of combinations sets of in vitro drug concentration and duration of exposure parameters is needed to recapitulate the in vivo concentration-time curve (FIGS. 11B, D). Therefore, a plurality of combinations of the concentration and duration for incubation may be selected for each identified substance. Based on the inventors' findings, the in vitro parameters should be selected to satisfy the conditions as stated in table 1.

Numerous treatment regimens include several administrations during the treatment cycle. For example, capecitabine monotherapy regimen includes 2 daily oral administrations of capecitabine for 2 weeks, 28 in total. To imitate clinical conditions cells in the in vitro assay can be repeatedly exposed to the active substance(s) of drugs of the evaluated regimen during assay time. If it is possible to select in vitro assay time that exceeds the treatment cycle time, then all administrations of the treatment cycle can be included in the assay. If the selected in vitro assay time is shorter than treatment cycle, then all administrations of the treatment cycle that fit the selected in vitro assay time can be included in the assay. In vitro assay time can be limited due to technical limitations e.g. small number of cells for evaluation of phenotypical changes, overgrowth of the cells etc., due to speed up the assay process or other reasons. For example, if total in vitro assay time is selected to be 72 hours and the evaluated treatment regimen is capecitabine monotherapy, then 5-fluorouracil can be added 6 times during the assay.

The cornerstone of the current cancer treatment is the combinational therapy. Differences in mechanism of action of cancer drugs lead to synergism, additive or potentiating effects. Therefore, to imitate clinical conditions it is important to evaluate if the drugs of a particular treatment regimen are present simultaneously in the body and utilize such data in the assay. To do so time of drug presence of its administration can be evaluated, and several reference parameters can be used.

The concentration-time curve of each active substance can be split into 2 sections (FIG. 12). First section is the timeframe from zero time point to the time Tmax where concentration reaches its maximum Cmax. If the drug is administered by infusion Tmax is the timepoint of the end of infusion. Second section is the timeframe during which drug concentration drops due to metabolism and excretion. The drop in concentration of an active substance can be described by half-life time parameter T½, which describes the time need for concentration to drop from Cmax to the half value of Cmax. Based on the inventors' findings, the clinical time of presence Tpr of an active substance should be selected in the range between the sum of Tmax and 0.1·T½ and the sum of Tmax and 10·T½.

The simultaneous presence of active substance(s) in the body can be taken into account and converted to appropriate concentrations, durations and time points of addition according to the method of the second aspect.

The time point of addition of the first substance of the first administration in a sequence of a treatment regimen is considered as a zero-time point of in vitro assay.

Preferably, the selected concentration(s) satisfy the conditions as defined in Table 1. The selected duration(s) may satisfy the conditions as defined in Table 1. The selected time point(s) of addition may satisfy the conditions as defined in Table 1. Most preferably, all of the aforementioned selections satisfy the conditions as defined in Table 1.

If the timepoint of an administration Ta in the treatment regimen is less than the sum of the timepoint of a previous administration and time of presence of an active substance of this previous administration then the substances of the administration and the substance of the previous administration are considered to be present simultaneously in the body. Therefore, in the vitro assay the active substances of the evaluated administration should be added to the cells in the timeframe between the selected time point of addition of the active substance of the previous administration and the sum of selected time point of addition of the active substance of the previous administration and selected duration of exposure of the active substance of the previous administration (FIG. 13, Table 1).

If the timepoint of an administration Ta in the treatment regimen is more than the sum of the timepoint of a previous administration and time of presence of an active substance of this previous administration then the substances of the administration and the substance of the previous administration are considered not to be present simultaneously in the body.

Therefore, in the vitro assay the active substances of the evaluated administration should be added to the cells in the timeframe between the sum of selected time point of addition of the active substance of the previous administration and selected duration of exposure of the active substance of the previous administration and selected point of addition of active substance(s) of the next administration (FIG. 13, Table 1). If the evaluated administration is the last one in the sequence then in the vitro assay the active substances of the evaluated administration should be added to the cells in the timeframe between the sum of selected time point of addition of the active substance of the previous administration and total time of the experiment Ttot.

Preferably, the selection of in vitro culture parameters is performed using the method of the second aspect.

Culturing the tumor cells may be performed in 2D cell culture, suspension cell culture or 3D cell culture including tumor organoids or their combination with stromal and immune cells. The cells may be cell lines or primary cells, preferably primary cells. The tumor cells may be preferably derived from an individual patient afflicted with a tumor disease, in which case the method may be performed to evaluate the efficacy of a chemotherapy, targeted therapy, immunotherapy cancer or their combinational drug treatment regimen in the treatment of said tumor disease.

The phenotypical change may be a change in the number of cells. Preferably, the phenotypical change may be cell proliferation or cell death. Various methods well known in the art exist for determining cell proliferation and cell death, including BrdU-assays, Ki67 immunostaining, live cell counting by various methods etc.

The drug treatment regimen is preferably considered effective when this regimen inhibits cell proliferation, stops cell proliferation or causes cell death.

### Algorithm for Selecting In Vitro Culture Parameters

In a second aspect, the present invention provides a method for selecting in vitro culture parameters for a method according to the first aspect, comprising the steps of:
a. selecting a drug treatment regimen to be evaluated;
b. selecting a total in vitro assay time Tₜₒₜ;
c. identifying the sequence [1, . . . , l] and time T_{a in vivo}ⁱ ([T_{a in vivo}¹, . . . , T_{a in vivo}^{l}]) of drug administrations included in said drug treatment regimen to be evaluated during Tₜₒₜ;
d. identifying the active substances Sₖⁱ ([S₁ⁱ, . . . , S_{K}ⁱ]) relevant for each identified administration i;
e. identifying clinical half life time T_{1/2[k]}ⁱ ([T_{1/2[1]}ⁱ. . . , T_{1/2[K]}ⁱ]) for each active substance Sₖⁱ for each administration i;
f. identifying clinical time T_{max[k]}ⁱ ([T_{max[1]}ⁱ, . . . , T_{max[K]}ⁱ]) for each active substance Sₖⁱ when concentration of this active substance reaches its maximum;
g. identifying clinical time of presence T_{pr[k]}ⁱ ([T_{pr[1]}ⁱ, . . . , T_{pr[K]}ⁱ]) for each active substance Sₖⁱ when this active substance is present in plasma in the range between T_{max[k]}ⁱ+1.0·T_{1/2[k]}ⁱ and T_{max[k]}ⁱ+10·T_{1/2[k]}ⁱ;
h. determining whether there is simultaneous presence of each identified active substance Sₖⁱ and any of the active substance(s) S_{y}^{x} of the preceding administration(s) and current administration i using the following rules:
   a. if T_{a in vivo}ⁱ−T_{a in vivo}^{x}≥T_{pr[y]}^{x} then the substances are considered not to be present simultaneously in the body;
   b. if T_{a in vivo}ⁱ−T_{a in vivo}^{x}<T_{pr[y]}^{x} then the substances are considered to be present simultaneously in the body;
i. identifying the in vivo area under the curve AUC_{in vivo[k]}ⁱ for the period Tₜₒₜ for each active substance Sₖⁱ for each administration i, based on clinical data for the selected regimen;
j. identifying the in vivo maximal concentration C_{max in vivo[k]}ⁱ for each active substance Sₖⁱ for each administration i, based on clinical data for the selected regimen;
k. for each identified active substance Sₖⁱ, selecting at least one combination (when applicable: several combinations 1, . . . N) of duration T_{ex[k][n]}ⁱ of exposure for in vitro assay and concentration C_{in vitro [k][n]}ⁱ for in vitro assay, to satisfy the following rules:

$$a . 0.01 \cdot {C}_{max in vivo [ k ]}^{i} \leq {C}_{in vitro [ k ] [ 1 ]}^{i} \leq 3 \cdot {C}_{max in vivo [ k ]}^{i} ;$$

$$b . \frac{0.2 \cdot {AUC}_{in vivo [ k ]}^{i}}{{C}_{in vitro [ k ] [ 1 ]}^{i}} \leq {T}_{ex [ k ] [ 1 ]}^{i} \leq \frac{3 \cdot {AUC}_{in vivo [ k ]}^{i}}{{C}_{in vitro [ k ] [ 1 ]}^{i}} ;$$

l. optionally: for each identified active substance Sₖⁱ, if C_{in vitro [k][1]}ⁱ·T_{ex[k][1]}ⁱ<3·AUC_{in vivo[k]}ⁱ then additional combinations of duration T_{ex[k][n]} of exposure for assay and concentration C_{in vitro [k][n]}ⁱ for assay are identified to satisfy the following rules:

0.01·C_{max in vivo[k]}ⁱ≤C_{in vitro [k][n]}ⁱ≤3·C_{max in vivo[k]}ⁱ; a.

0.2·AUC_{in vivo[k]}ⁱ≤AUC_{in vitro[k]}ⁱ≤3·AUC_{in vivo[k]}ⁱ, where AUC_{in vitro[k]}ⁱ=AUC_{in vitro[k][1]}ⁱ+ . . . +AUC_{in vitro[k][N]}ⁱ and AUC_{in vitro[k][n]}ⁱ=C_{in vitro [k][n]}ⁱ·T_{ex[k][n]}ⁱ; b.
m. for all identified administrations and all identified active substances sum of all durations of exposure for assay must satisfy the following criterion: ΣT_{ex[k][n]}ⁱ≤Tₜₒₜ;
n. for each identified active substance Sₖⁱ relevant for the first identified administration time point of addition in vitro T_{a in vitro [k]}ⁱ for assay must satisfy the following rule: 0≤T_{a in vitro[k]}¹≤T_{max[k]}¹; and
o. for each identified active substance Sₖⁱ relevant for any administration except the first one selecting time point of addition in vitro T_{a in vitro[k]}ⁱ for assay must satisfy the following rules:
   a. if identified active substance Sₖⁱ and any of the active substance(s) S_{y}^{x} of the preceding administration(s) and current administration l are present simultaneously in the body then T_{a in vitro[y]}^{x}≤T_{a in vitro[k]}ⁱ≤T_{a in vitro[y]}^{x}+ΣₙT_{ex[y][n]}^{x};
   b. if identified active substance Sₖⁱ and any of the active substance(s) S_{y}^{x} of the preceding administration(s) and current administration l are not present simultaneously in the body then T_{a in vitro[y]}^{x}+ΣₙT_{ex[y][n]}^{x}≤T_{a in vitro[k]}ⁱ<Tₜₒₜ.

Tₜₒₜ is based on clinical time of treatment of a patient with the chosen treatment regimen and the appropriate time for cell culture growth to prevent overgrowth and spontaneous death of the cells in vitro, and may represent a practical compromise due to the limitations of the in vitro culture.

Tₜₒₜ may be the time needed for the tumor cells to increase by at least 1.2-fold in cell numbers in culture. Preferably, Tₜₒₜ≤72 h for practical reasons. Most preferably, Tₜₒₜ is about 48-72 h.

Preferably, the selected time point(s) of addition satisfy the conditions as defined in Table 1.

### Preferred Embodiments of the Method of the First Aspect

In a preferred embodiment, the selected drug treatment regimen is FOLFOX, and the selected in vitro culture parameters satisfy the following criteria:

| | | Substance | Duration | Time point |
|---|---|---|---|---|
| Sequence | | concentration | of exposure | of addition |
| number | Substance | range (μM) | range (h) | range (h) |
| 1 | Oxaliplatin | 0.2-10.8 | 0.13-24 | 0-0 |
| 2 | 5-fluorouracil | 15-400 | 0.03-5 | 0.13-24 |
| 3 | 5-fluorouracil | 0.45-16.5 | 2-72 | 0.13-29 |

In another preferred embodiment, the selected drug treatment regimen is XELOX, and the selected in vitro culture parameters satisfy the following criteria:

| | | Substance | Duration | Time point |
|---|---|---|---|---|
| Sequence | | concentration | of exposure | of addition |
| number | Substance | range (μM) | range (h) | range (h) |
| 1 | Oxaliplatin | 0.5-41 | 0.13-24 | 0-0 |
| 2 | 5-fluorouracil | 0.5-41 | 0.15-12 | 0.13-36 |
| 3 | 5-fluorouracil | 0.5-41 | 0.15-12 | 0.28-48 |
| n | 5-fluorouracil | 0.5-41 | 0.15-12 | 0.13 + |
| | | | | (n − 2)*0.15 |
| | | | | to 36 + 12* |
| | | | | (n − 2) |

In yet another preferred embodiment, the selected drug treatment regimen is FOLFIRI, and the selected in vitro culture parameters satisfy the following criteria:

| | | Substance | Duration | Time point |
|---|---|---|---|---|
| Sequence | | concentration | of exposure | of addition |
| number | Substance | range (μM) | range (h) | range (h) |
| 1 | CPT-11 | 0.1-10.89 | 0.35-72 | 0-0 |
| 1 | SN-38 | 0.002-0.266 | 0.35-72 | 0-2.3 |
| 2 | 5-fluorouracil | 15-400 | 0.03-5 | 0.35-72 |
| 3 | 5-fluorouracil | 0.45-16.5 | 2-72 | 0.35-72 |

In a further preferred embodiment, the selected drug treatment regimen is capecitabine monotherapy, and the selected in vitro culture parameters satisfy the following criteria:

| | | Substance | Duration | Time point |
|---|---|---|---|---|
| Sequence | | concentration | of exposure | of addition |
| number | Substance | range (μM) | range (h) | range (h) |
| 1 | 5-fluorouracil | 0.5-41 | 0.15-12 | 0-0 |
| 2 | 5-fluorouracil | 0.5-41 | 0.15-12 | 0.15-24 |
| 3 | 5-fluorouracil | 0.5-41 | 0.15-12 | 0.3-36 |
| n | 5-fluorouracil | 0.5-41 | 0.15-12 | 0.15 + |
| | | | | (n − 2)*0.15 |
| | | | | to 24 + 12* |
| | | | | (n − 2) |

### Devices and Computer-Implemented Aspects

In a third aspect, the present invention provides a device adapted to perform the method of the first aspect, comprising:
a. a programmable control unit (7) for executing device functions to perform the method;
b. an incubator unit (8) for in vitro tumor cell culture;
c. a storage unit (4) for the active compound(s);
d. a liquid handling unit (2) for administering the active compound(s) to cultured tumor cells in accordance with the method; and
e. a detector for determining the phenotypical changes of tumor cells (6) due to effect of the active substance(s).

In a fourth aspect, there is provided a computer program product comprising instructions to cause the device of the third aspect to execute the steps of the method of the first aspect.

In fifth aspect, there is provided a computer-readable data carrier having stored thereon the computer program product of the fourth aspect.

In a fifth aspect, there is provided a computer-implemented method of the second aspect.

In a sixth aspect, there is provided a data processing device comprising means for carrying out the steps of the method of the fifth aspect.

In a seventh aspect, there is provided a computer program product comprising instructions which, when the program is executed by a computer, cause the computer to carry out the steps of the method of the fifth aspect.

In an eighth aspect, there is provided a computer-readable data carrier having stored thereon the computer program product of the seventh aspect.

In a ninth aspect, there is provided a data carrier signal carrying the computer program product of the seventh aspect.

TABLE 1
Criteria for a method for selecting parameters for the in vitro assay of
the first aspect mimicking in vivo pharmacology of a treatment regimen.

| Parameter | Criteria | |
|---|---|---|
| Sequence | 1 (The first | 2, . . . , I (From the second one to the last one) |
| number | one) | |
| Active | Any identified | Any identified active substance of the evaluated administration |
| substance | active | |
| identified | substance of | |
| | the first | |
| | administration | |
| Substance | Should exceed 0.01x in vivo maximum concentration | |
| minimum | | |
| concentration | | |
| Substance | Should not exceed 3x in vivo maximum concentration | |
| maximum | | |
| concentration | | |
| Minimum | Should not be less than product of 0.2 and area under curve in the body divided by selected | |
| duration of | concentration | |
| exposure, h | | |
| Maximum | Should not exceed product of 15 and minimum duration of exposure | |
| duration of | | |
| exposure, h | | |
| Earliest time | 0 | If the evaluated active substance and another active substance of current |
| point of | | comparison are present simultaneously in the body, then: |
| addition*, h | | Earliest time point of addition of the evaluated active substance is equal to |
| | | the selected time point of addition of the other active substance of current |
| | | comparison |
| | | If the evaluated active substance and another active substance of current |
| | | comparison are not present simultaneously in the body, then: |
| | | Earliest time point of addition of the evaluated active substance should be |
| | | equal to the sum of selected time point of addition of the other active |
| | | substance of current comparison and selected duration of exposure of the |
| | | other active substance of current comparison |
| Latest time | The time when | If the evaluated active substance and another active substance of current |
| point of | concentration | comparison are present simultaneously in the body, then: |
| addition*, h | of this active | Latest time point of addition of the evaluated active substance should be |
| | substance | equal to the sum of selected time point of addition and selected duration of |
| | reaches its | exposure of the other active substance of current comparison |
| | maximum in | If the evaluated active substance and the other active substance of current |
| | vivo | comparison are not present simultaneously in the body, then: |
| | | Latest time point of addition of the evaluated active substance should be |
| | | equal to the minimum time point of addition of active substance(s) of the |
| | | next administration(s) and in any case less than total time of the in vitro |
| | | experiment |

*The range of possible time points of addition is determined based on the fact whether there is simultaneous presence of the evaluated active substance of current administration and any of the active substance(s) of the preceding administration(s) and current administration. Each comparison results into a separate range. Then the resulting range of possible time points of addition of the evaluated active substance is determined by intersection of all calculated ranges.

The term “comprising” is to be interpreted as including, but not being limited to. All references are hereby incorporated by reference. The arrangement of the present disclosure into sections with headings and subheadings is merely to improve legibility and is not to be interpreted limiting in any way, in particular, the division does not in any way preclude or limit combining features under different headings and subheadings with each other.

### EXAMPLES

The following examples are not to be regarded as limiting.

### Example 1

### Identifying Administration Sequence, Active Substances and Combination of the Time Points, Concentrations, and Durations of Exposure for the mFOLFOX6 Protocol

Step 1. Identification of Sequence and Time of Drug Administrations of mFOLFOX6 Protocol

According to National Comprehensive Cancer Network Clinical Practice Guidelines in Oncology: p Colon Cancer. V. 4.202020 (website: nccn.org/professionals/physician_gls/PDF/colon.pdf) mFOLFOX6 protocol treatment cycle based on the sequence of the following administrations presented in Table 2.

TABLE 2
Sequence and time of drug administrations
of mFOLFOX6 protocol

| Sequence number | Administration | Time |
|---|---|---|
| 1-2 | Oxaliplatin Infusion | 0 h |
| 1-2 | Leucovorin Infusion | 0 h |
| 3 | 5-Fluorouracil bolus | 2 h |
| 4 | 5-Fluorouracil continuous infusion | 2 h 2 m[13] |

Step 2. Identification of at Least One Active Substance Relevant for Each Identified Administration.

According to pharmacological data of each administered drug available elsewhere (e.g. website: drugbank.ca), active substances were identified and presented in Table 3.

TABLE 3
Identified active substances relevant
for each identified administration

| Administration | Active substance |
|---|---|
| Oxaliplatin Infusion | Oxaliplatin |
| Leucovorin Infusion | Leucovorin |
| 5-Fluorouracil bolus | 5-Fluorouracil |
| 5-Fluorouracil continuous infusion | 5-Fluorouracil |

Leucovorin does not possess direct cytotoxic or cytostatic effect in accordance to NCI Database (website: cancer.gov/about-cancer/treatment/drugs/leucovorincalcium) and was excluded from the sequence.

Step 3. Identification of Time Points of Addition, Concentrations, and Durations of Exposure of Substances.

Pharmacokinetics parameters for all substances presented in Table 3 were identified based on published data [13-16]. Substances of administration 1 (Oxaliplatin) and 2 (5-Fluorouracil) present simultaneously in the body, so time point of addition of 2 (5-Fluorouracil) was selected in a timeframe of selected incubation time for 1 (Oxaliplatin) [14]. Substances of administration 2 (5-Fluorouracil) and 3 (5-Fluorouracil) also present simultaneously in the body, so time point of addition of 3 (5-Fluorouracil) was selected in a timeframe of selected duration of exposure for 2 (5-Fluorouracil) [13]. All identified parameters for the assay according to criteria stated in Table 1 are presented in Table 4.

TABLE 4
Identified time points of addition, concentrations,
and durations of exposure of substances.

| Parameter | Value | | |
|---|---|---|---|
| Sequence number | 1 | 2 | 3 |
| Active substance | Oxaliplatin | 5-Fluorouracil | |
| Cmax, μM | 3.6 | 272.8* | 5.5 |
| Area under curve, μM· | 6.8 | 82.1* | 156.4 |
| Minimum concentration, μM | 0.036 | 2.7 | 0.055 |
| Maximum concentration, μM | 10.8 | 818.4 | 16.5 |
| Selected concentration, μM | 10 | 50 | 10 |
| Minimum duration of exposure, h | 0.14 | 0.33 | 3.13 |
| Maximum duration of exposure, h | 2.04 | 4.93 | 46.92 |
| Selected duration of exposure, h | 1 | 1 | 40 |
| Earliest time point of addition, h | 0 | 0 | 1 |
| Latest time point of addition, h | 2 | 1 | 1.5 |
| Selected time point of addition, h | 0 | 0.5 | 1.2 |

*[13]Calculated to a dose of 400 mg/m² assuming linear kinetics

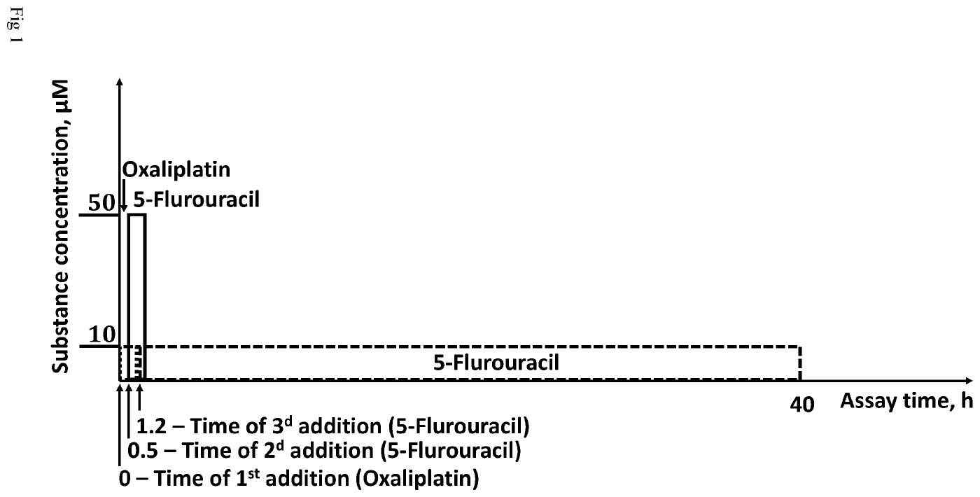

As result assay protocol for mFOLFOX6 protocol has been created (FIG. 1).

### Example 2

### Influence of Combinations of Assay Parameters on Performance of the Method of Evaluation of the mFOLFOX6 Regimen Efficacy

A patient was diagnosed with colon cancer and mFOLFOX6 chemotherapeutic treatment was prescribed. To obtain primary organoid culture of colon cancer cells a sample of resected lung metastasis was used. Tissue was cut into small fragments and placed immediately into MACS tissue storage solution (Miltenyi Biotec, Germany) and stored for no more than 24 hours at 4° C. Then, the tissue fragments were transferred to a tube for tissue homogenization gentleMACS C Tube (Miltenyi Biotec, Germany) and enzyme cocktail from Tumor Dissociation Kit human (Miltenyi Biotec, Germany) consisting of 2.2 ml of DMEM/F-12 culture medium (Thermo Fisher Scientific, USA), 100 μl of Enzyme H solution (Miltenyi Biotec, Germany), 50 μl of Enzyme R solution (Miltenyi Biotec, Germany) and 12.5 μl of Enzyme A solution (Miltenyi Biotec, Germany) was added to the same tube. Then the tube was tightly closed with a lid and placed in gentleMACS Octo Dissociator (Miltenyi Biotec, Germany). For tissue dissociation, the “37C_h_TDK_3” program was used. After the end of the program, the tube was removed from the dissociator. The resulting suspension was centrifuged at 300 g for 10 minutes. The supernatant was removed and the pellet was resuspended in 10 ml of DPBS (Thermo Fisher Scientific, USA). Then the suspension was re-centrifuged with similar parameters, the supernatant was also removed and the pellet was resuspended in DMEM/F-12 culture medium (Thermo Fisher Scientific, USA). Then the tube with the suspension was placed on ice and the suspension was mixed with Matrigel Growth Factor Reduced (GFR) Basement Membrane Matrix (Corning, USA) in the ratio 1:2. Then 50 μl Drops of the resulting suspension in the extracellular matrix were transferred into the wells of a 24-well culture plate (TPP, Switzerland) and placed into cell culture incubator (37° C., 5% CO2) for 20 minutes for solidification of the gel. Then, 750 μl of complete cell culture medium was added to each well and the plate was incubated in cell culture incubator (37° C., 5% CO2). The recipe of the complete cell culture medium used in this study was published previously[17]. Cell culture medium was replaced every 48 hours. Cells were inspected visually by inverted Primo Vert microscope (Carl Zeiss, Germany). Organoids were subcultured every 2 weeks with the help of TrypLE Express (Thermo Fisher Scientific, USA) as described previously[17].

To evaluate the efficacy of the mFOLFOX6 regimen two sets of parameters were selected (Table 5).

TABLE 5
Selected time points of addition, concentrations, and durations
of exposure of the substances for evaluation of the efficacy
of the mFOLFOX6 regimen using primary organoid culture.

| Parameter | Value | | |
|---|---|---|---|
| Sequence number | 1 | 2 | 3 |
| Active substance | Oxaliplatin | 5-Fluorouracil | |
| Cmax, μM | 3.6 | 272.8* | 5.5 |
| Area under curve, μM· | 6.8 | 82.1* | 156.4 |
| Minimum concentration, μM | 0.036 | 2.7 | 0.055 |
| Maximum concentration, μM | 10.8 | 818.4 | 16.5 |

Set 1

| | | | |
|---|---|---|---|
| Selected concentration, μM | 10 | 50 | 10 |
| Minimum duration of exposure, h | 0.14 | 0.33 | 3.13 |
| Maximum duration of exposure, h | 2.04 | 4.93 | 46.92 |
| Selected duration of exposure, h | 1 | 1 | 40 |
| Earliest time point of addition, h | 0 | 0 | 1 |
| Latest time point of addition, h | 2 | 1 | 1.5 |
| Selected time point of addition, h | 0 | 0.5 | 1.2 |

Set 2

| | | | |
|---|---|---|---|
| Selected concentration, μM | 1 | 35 | 1 |
| Minimum duration of exposure, h | 1.36 | 0.47 | 31.28 |
| Maximum duration of exposure, h | 20.40 | 7.04 | 469.20 |
| Selected duration of exposure, h | 1 | 24 | 24 |
| Earliest time point of addition, h | 0 | 0 | 1 |
| Latest time point of addition, h | 2 | 1 | 24.5 |
| Selected time point of addition, h | 0 | 0.5 | 1.2 |

As seen from Table 5 Set 2 includes parameters, that violate criteria stated in Table 1.

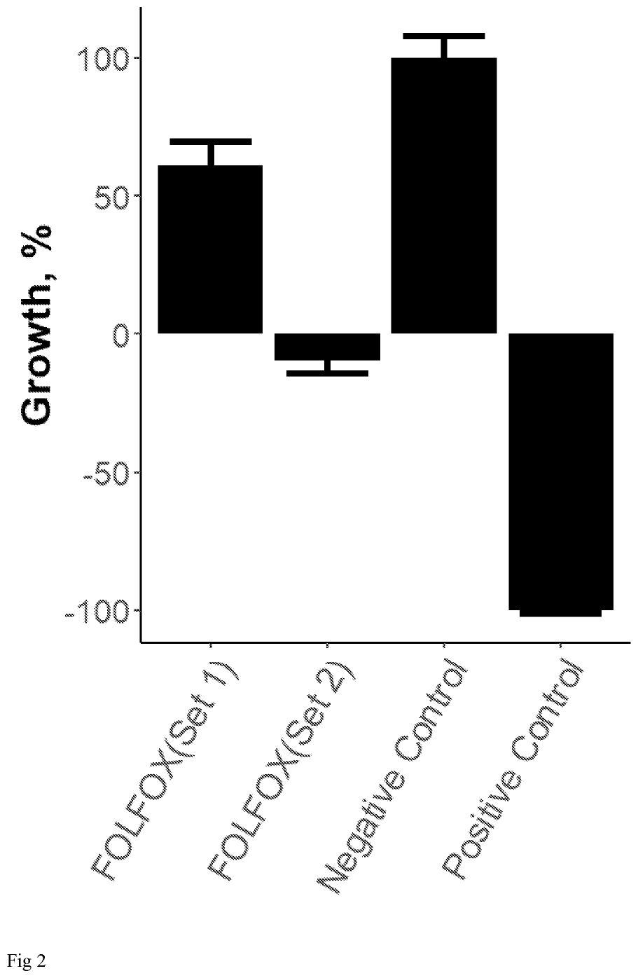

Organoids were diluted in Matrigel Growth Factor Reduced (GFR) Basement Membrane Matrix (Corning, USA) and seeded into 96-well plate (TPP, Switzerland). In 24 h cell culture medium was replaced with the fresh medium and organoids were cultured with addition of each identified active substances at certain time points in accordance to the sequence and incubated in the presence of the concentrations of said active substances for a certain durations of exposure identified for Set 1 and Set 2 and presented in Table 5. Relative number of cells was measured with MTS assay CellTiter 96 Aqueous One Solution Cell Proliferation Assay kit (Promega, USA) according to the manufacturer's instructions (FIG. 2).

As seen on FIG. 2 two Sets of parameters used for evaluation of the efficacy of mFOLFOX6 regimen lead to controversial result. According to clinical data patient had a progression after mFOLFOX6 indicating that tumor cells were resistant to mFOLFOX6 regimen. Given this data we postulate that fulfillment of the criteria stated in Table 1 is necessary for reliable evaluation of the efficacy of a. drug treatment regimen.

### Example 3

### Comparison of the Performances of Different Methods for Evaluating the Efficacy of a Drug Treatment Regimens

A patient was diagnosed with colon cancer and mFOLFOX6 chemotherapeutic treatment was prescribed. To obtain primary organoid culture of colon cancer cells a sample of resected lung metastasis was used. Organoids were prepared accordingly to Example 2. To evaluate the efficacy of the mFOLFOX6 regimen several methods were used. First method included sequential addition of substances at defined time points of addition to deliver defined concentrations in the culture medium and incubation of organoids with substances for defined durations of exposure accordingly to the criteria presented in Table 1. The second method was based on addition of serial dilutions of the same substances, incubation of organoids for 72 hours and identification of half growth inhibitory concentrations of the said substances. Third method was based on incubation of organoids with clinically relevant concentrations of substances for 4 hours accordingly to Romero-Calvo et al[11].

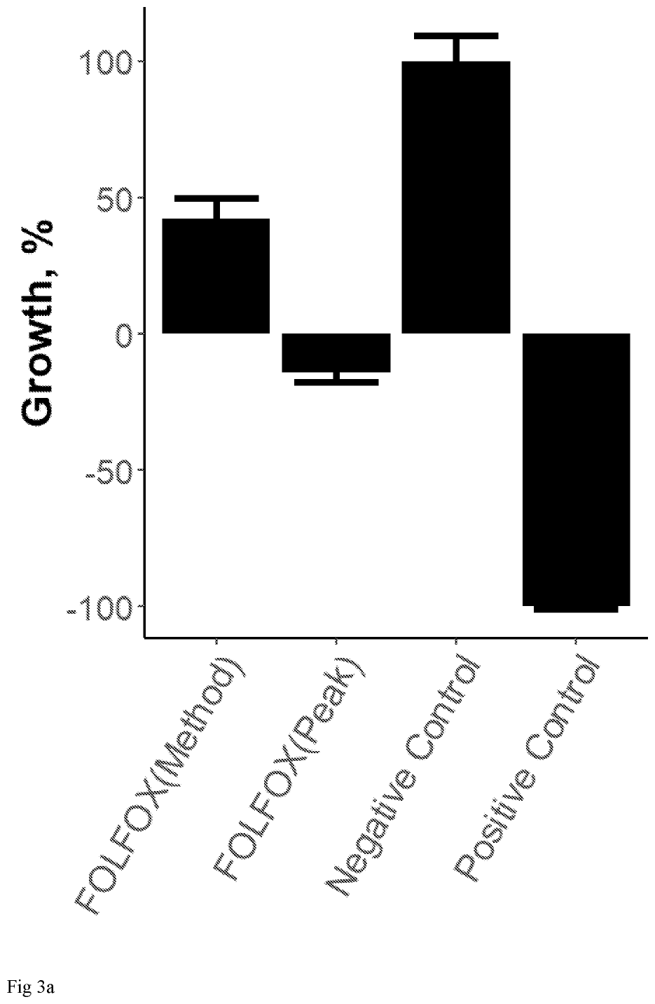

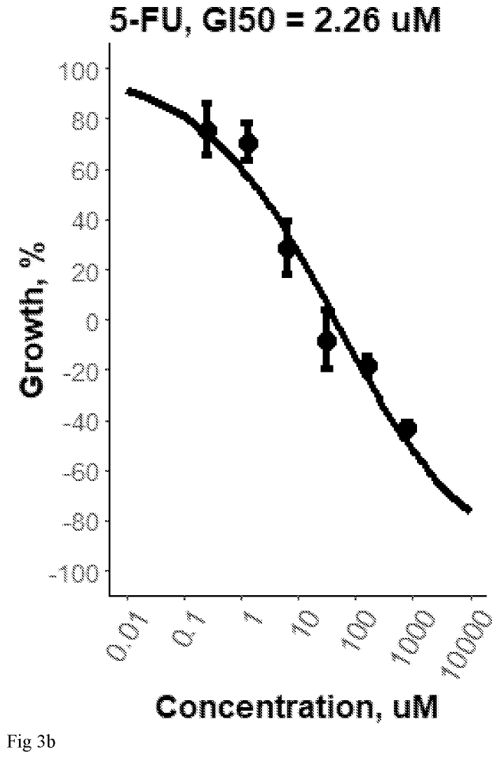

Cells were cultured and viability in was measured accordingly to Example 2 (FIG. 3).

As seen at FIG. 3 b, half growth inhibitory concentration for 5-fluorouracil was identified at 2.26 μM. This concentration is more that 150× lower than maximum concentration observed after bolus injection of the drug at 400 mg/m² dose[18]. This means that observed GI₅₀ concentration can be considered as clinically relevant and the examined colon cancer organoids can be considered as sensitive to 5-fluorouracil. The method described in Romero-Calvo et al[11] has also revealed susceptibility of the colon cancer organoids to substances of mFOLFOX6 regimen. Method of evaluating the efficacy of a drug mFOLFOX6 treatment regimen based on criteria presented in Table 1 resulted in opposite conclusions—colon cancer cells were still growing. According to clinical data patient had a progression after mFOLFOX6 indicating that tumor cells were resistant to mFOLFOX6 regimen. Given this data we postulate that evaluation of the efficacy of drug treatment regimen based on criteria presented in Table 1 could provide more precise results.

### Example 4

### In Vitro Comparison of the Efficacy of Clinically Equal Drug Treatment Regimens

XELOX and mFOLFOX6 treatment regimens considered as clinically similar in terms of efficacy[19,20]. To evaluate performance of the described method for evaluation of the efficacy of drug treatment regimen based on criteria presented in Table 1 we performed comparative study with the method described in Romero-Calvo et al[11] and presented in Example 3.

For the method described in Example 2 parameters of mFOLFOX6 and XELOX regimens namely substances, time points of addition, concentrations and durations of exposure were selected accordingly to criteria presented in Table 1. For XELOX regimen oxaliplatin and 5-fluorouracil were identified as active substances and selected parameters are presented in Table 6.

TABLE 6
Identified time points of addition, concentrations, and durations of
exposure of substances for mFOLFOX6 and XELOX treatment regimens.

| | |
|---|---|
| Parameter | Value |

mFOLFOX6

| | | | |
|---|---|---|---|
| Sequence number | 1 | 2 | 3 |
| Active substance | Oxaliplatin | | 5-Fluorouracil |
| Selected concentration, μM | 10 | 50 | 10 |
| Selected duration of exposure, h | 1 | 1 | 40 |
| Selected time point of addition, h | 0 | 0.5 | 1.2 |

XELOX

| Sequence number | 1 | 2 | 3 | 4 | 5 | 6 | 7 |
|---|---|---|---|---|---|---|---|
| Active substance | Oxaliplatin | 5-Fluorouracil | | | | | |
| Selected concentration, μM | 10 | 4.5 | 4.5 | 4.5 | 4.5 | 4.5 | 4.5 |
| Selected duration of exposure, h | 1.5 | 2 | 2 | 2 | 2 | 2 | 2 |
| Selected time point of addition, h | 0 | 1.5 | 3.5 | 25.5 | 27.5 | 49.5 | 51.5 |

*[13] Calculated to a dose of 400 mg/m² assuming linear kinetics;
**[14] Calculated to a dose of 130 mg/m² assuming linear kinetics.

For the comparison method described in Romero-Calvo et al[11] clinically relevant concentrations of the said substances were selected and duration of exposure was set to 4 hours.

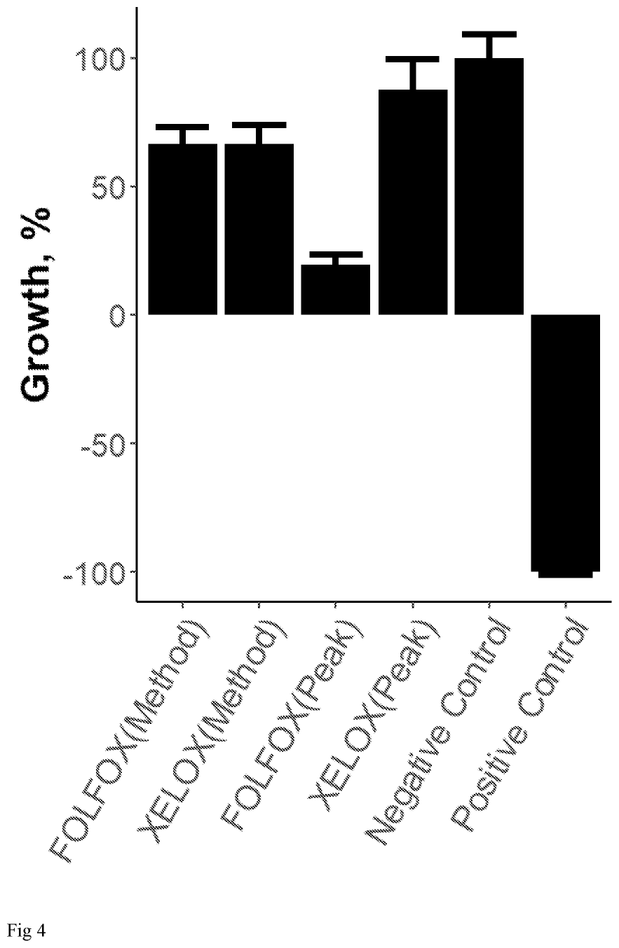

A patient was diagnosed with colon cancer and XELOX followed by mFOLFOX6 chemotherapeutic treatment was prescribed. To obtain primary organoid culture of colon cancer cells a sample of resected lung metastasis was used. Organoids were prepared accordingly to Example 2. Cells were cultured and relative number of cells was measured accordingly to Example 3 (FIG. 4).

As seen at FIG. 4 described approach resulted in statistically similar results for XELOX and mFOLFOX6 regiments despite the fact, that parameters used for the method for each regimen such as concentrations, times of addition and durations of exposure were different. In accordance to the results obtained using method described in Romero-Calvo et al[11] mFOLFOX6 regimen was more efficient than XELOX regimen. Given this data we postulate that evaluation of the efficacy of drug treatment regimen based on criteria presented in Table 1 could provide comparable results for clinically similar treatment regimens.

### Example 5

### Identification the Most Effective Drug Treatment Regimen

A patient was diagnosed with colon cancer and FOLFIRI chemotherapeutic treatment was prescribed. To obtain primary organoid culture of colon cancer cells a sample of resected liver metastasis was used. Organoids were prepared accordingly to Example 2. For the method described in Example 2 parameters of FOLFIRI, XELOX and Capecitabine regimens namely substances, time points of addition, concentrations and durations of exposure were selected accordingly to criteria presented in Table 1. For XELOX regimen oxaliplatin and 5-fluorouracil were identified as active substances. For FOLFIRI regimen 5-fluorouracil, CPT-11 and SN-38 were identified as active substances. For Capecitabine monotherapy regimen 5-fluorouracil was identified as active substance. Selected parameters are presented in Table 7.

TABLE 7
Identified time points of addition, concentrations, and durations of exposure of
substances for FOLFIRI, XELOX and Capecitabine monotherapy treatment regimens.

| | |
|---|---|
| Parameter | Value |

FOLFIRI

| | | | | |
|---|---|---|---|---|
| Sequence number | 1 | 2 | 3 | 4 |
| Active substance | CPT-11 | SN-38, nM | | 5-Fluorouracil |
| Selected concentration, μM | 3 | 100 nM | 50 | 10 |
| Selected duration of exposure, h | 6 | 6 | 1 | 40 |
| Selected time point of addition, h | 0 | 0 | 5 | 6 |

XELOX

| Sequence number | 1 | 2 | 3 | 4 | 5 | 6 | 7 |
|---|---|---|---|---|---|---|---|
| Active substance | Oxaliplatin | 5-Fluorouracil | | | | | |
| Selected concentration, μM | 10 | 4.5 | 4.5 | 4.5 | 4.5 | 4.5 | 4.5 |
| Selected duration of exposure, h | 1.5 | 2 | 2 | 2 | 2 | 2 | 2 |
| Selected time point of addition, h | 0 | 1.5 | 3.5 | 25.5 | 27.5 | 49.5 | 51.5 |

Capecitabine monotherapy

| | | | | | | |
|---|---|---|---|---|---|---|
| Sequence number | 1 | 2 | 3 | 4 | 5 | 6 |

| | |
|---|---|
| Active substance | 5-Fluorouracil |

| | | | | | | |
|---|---|---|---|---|---|---|
| Selected concentration, μM | 4.5 | 4.5 | 4.5 | 4.5 | 4.5 | 4.5 |
| Selected duration of exposure, h | 2 | 2 | 2 | 2 | 2 | 2 |
| Selected time point of addition, h | 0 | 2 | 24 | 26 | 48 | 50 |

*[13] Calculated to a dose of 400 mg/m² assuming linear kinetics;
**[14] Calculated to a dose of 130 mg/m² assuming linear kinetics.

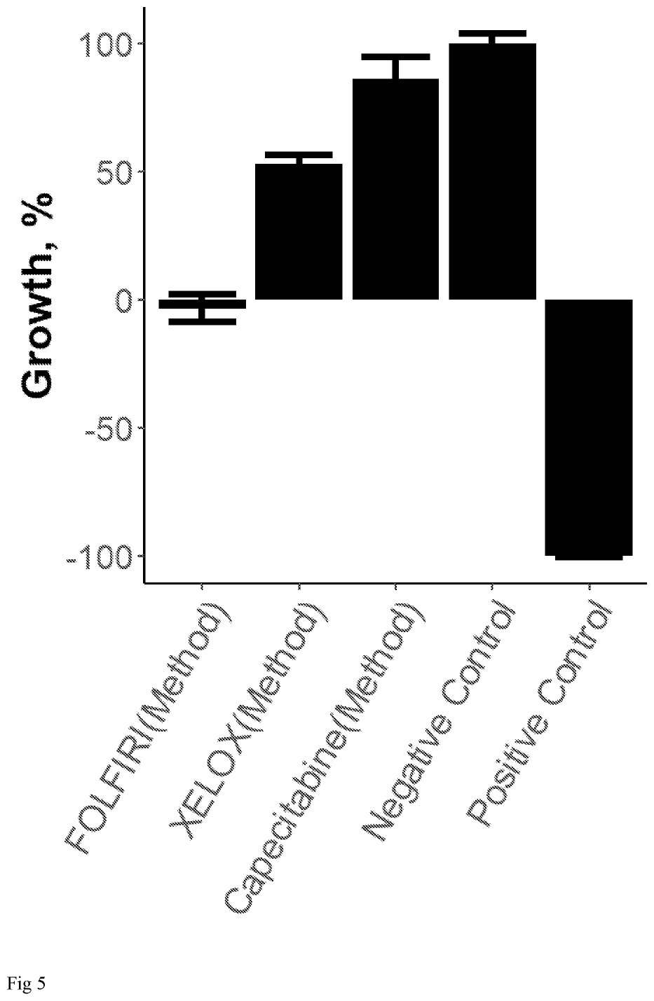

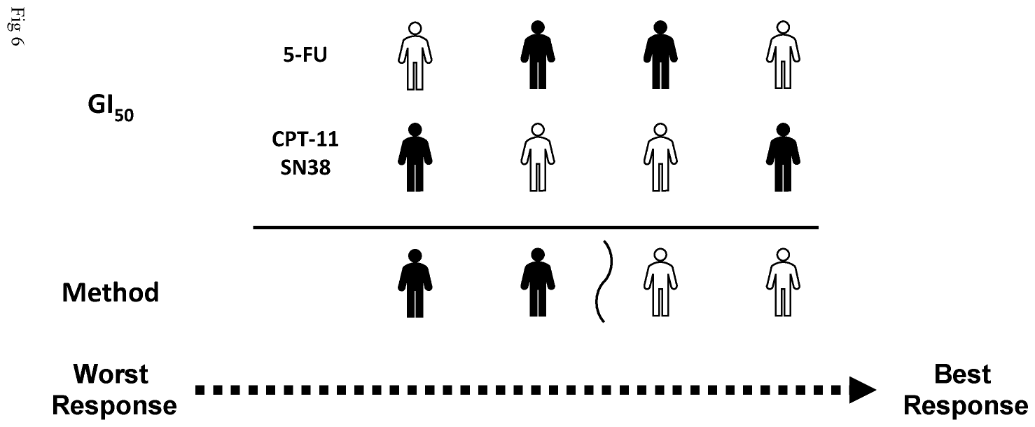

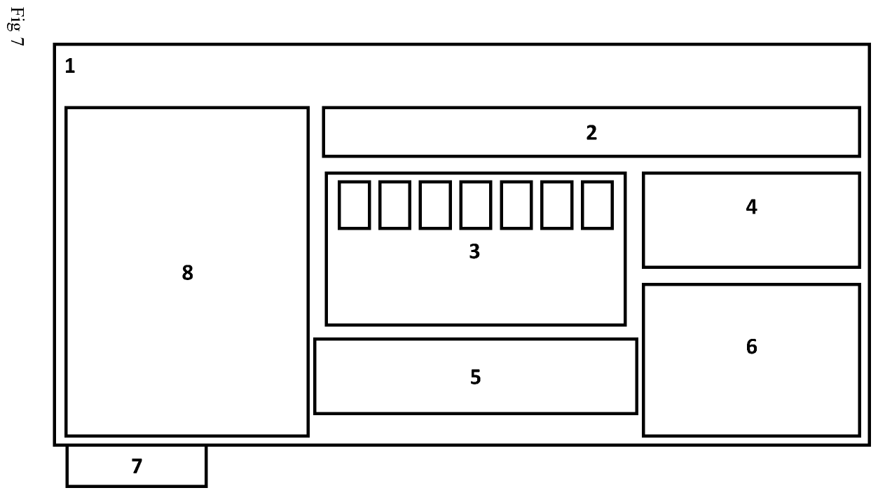

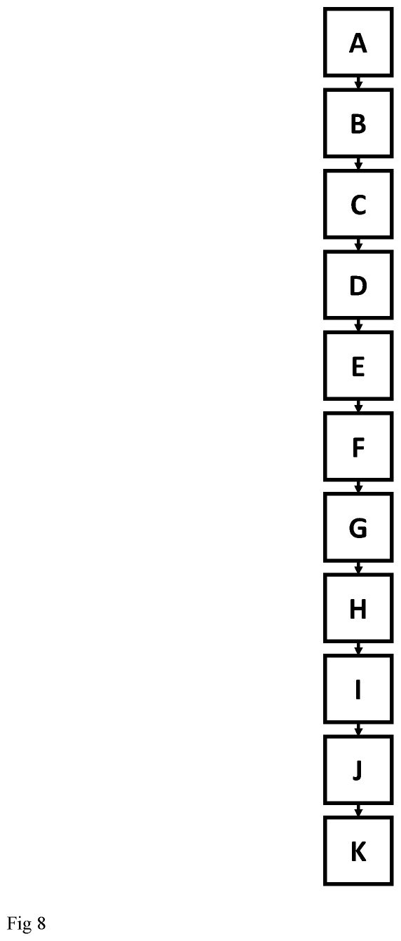

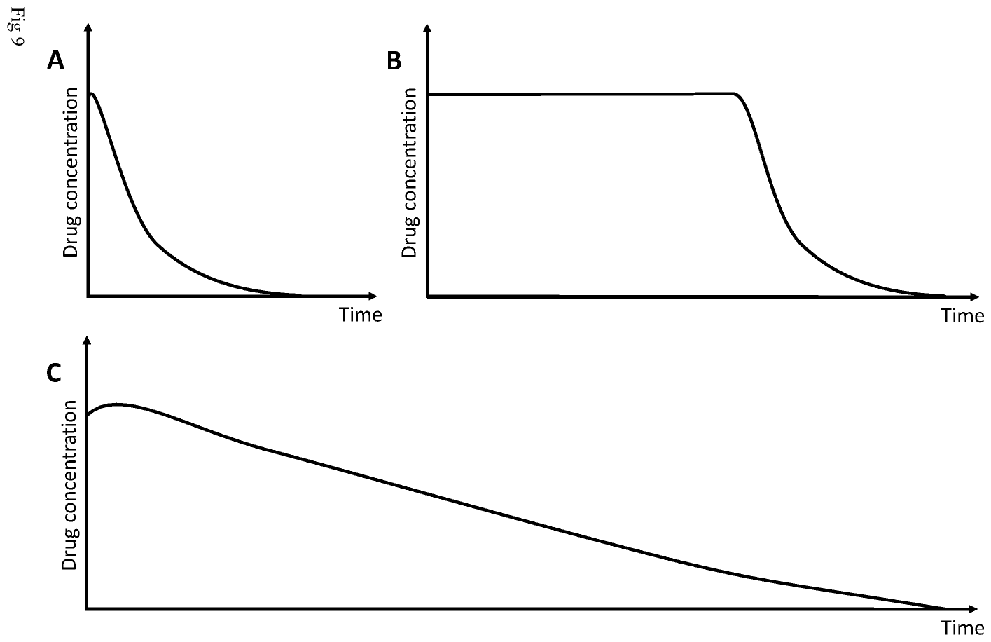

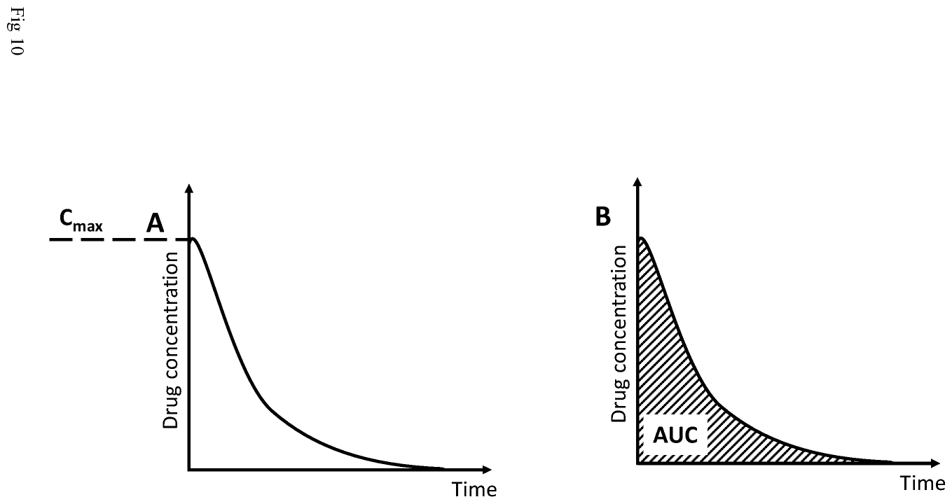

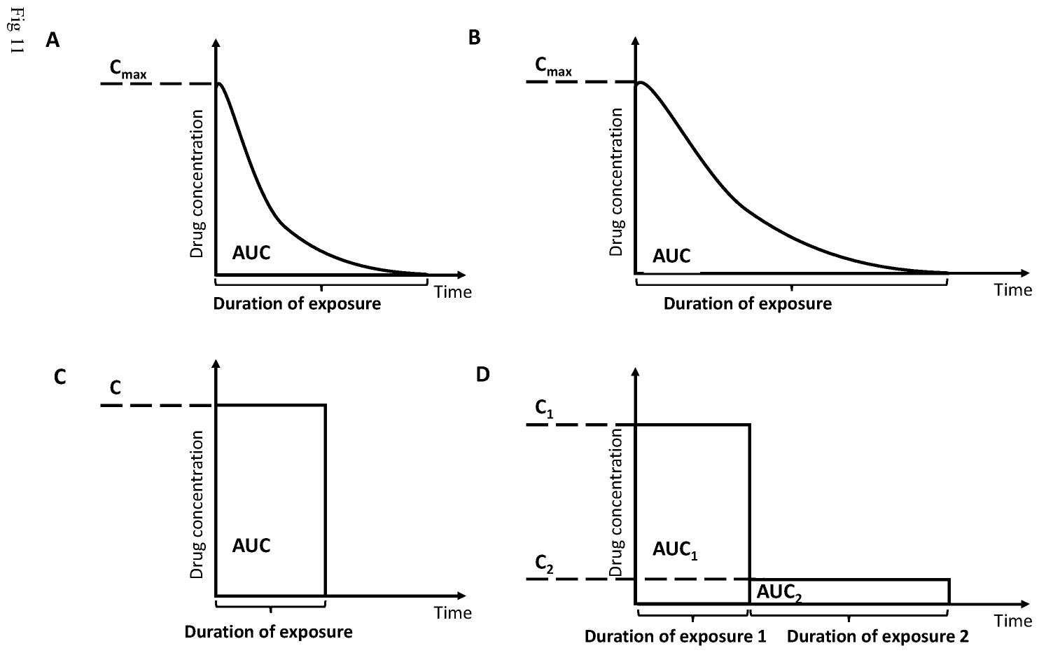

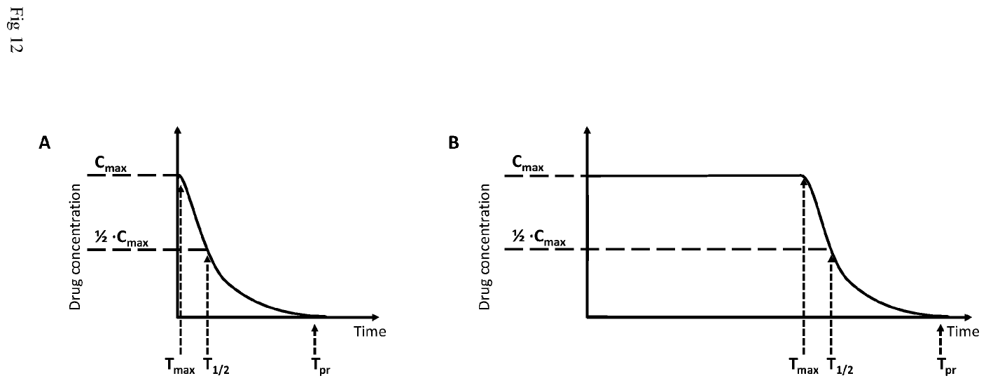

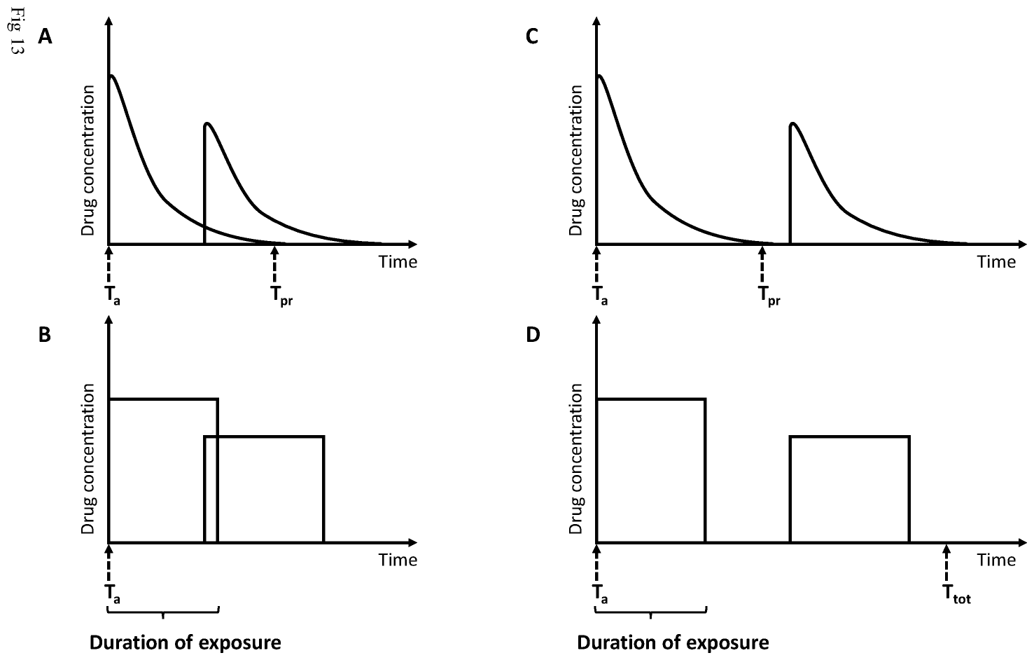

Cells were cultured and relative number of cells was measured accordingly to Example 3 (FIG. 5).

As seen in FIG. 5 only FOLFIRI regimen would be beneficial for the patient as the colon cancer cells were susceptible to this regimen. According to clinical data patient had a response after FOLFIRI regimen indicating that tumor cells were susceptible to this regimen in vivo. Given this data we postulate that evaluation of the efficacy of drug treatment regimen based on criteria presented in Table 1 could provide results comparable with clinic response.

### Example 6

### Comparison of the Clinical Response and In Vitro Results Using Different Methods for Evaluating the Efficacy of a Drug Treatment Regimens

Patients were diagnosed with colon cancer and 5-fluorouracil and irinotecan containing chemotherapeutic treatment was prescribed. To obtain primary organoid cultures of colon cancer cells the samples of resected metastasis were used. Organoids were prepared according to Example 2. For the method described in Example 2 parameters of FOLFIRI regimen namely substances, time points of addition, concentrations and durations of exposure were selected according to criteria presented in Table 1. For FOLFIRI regimen 5-fluorouracil, CPT-11 and SN-38 were identified as active substances. Selected parameters are presented in Table 7. The comparator GI₅₀ method was based on addition of serial dilutions of the same substances, incubation of organoids for 72 hours and identification of half growth inhibitory concentrations of the said substances. Patients were ordered according to the response of in vitro drug tests. As one can see in FIG. 6 the responders and non-responders could not be accurately predicted based on GI₅₀ test. In contrast, the method of the present invention allowed to predict clinical responders and non-responders based on in vitro test results.

### REFERENCES

1 Hammond W. A., Swaika A., Mody K. (2016) Pharmacologic resistance in colorectal cancer: a review. Ther. Adv. Med. Oncol. 8, 57-84.

2 Leto S. M., Trusolino L. (2014) Primary and acquired resistance to EGFR-targeted therapies in colorectal cancer: impact on future treatment strategies. J. Mol. Med. 92, 709-722.

3 Lièvre A., Bachet J. B., Le Corre D., Boige V., Landi B., Emile J. F., Côté J. F., Tomasic G., Penna C., Ducreux M., Rougier P., Penault-Llorca F., Laurent-Puig P. (2006) KRAS mutation status is predictive of response to cetuximab therapy in colorectal cancer. Cancer Res. 66, 3992-3995.

4 Paik S., Tang G., Shak S., Kim C., Baker J., Kim W., Cronin M., Baehner F. L., Watson D., Bryant J., Costantino J. P., Geyer C. E., Wickerham D. L., Wolmark N. (2006) Gene Expression and Benefit of Chemotherapy in Women With Node-Negative, Estrogen Receptor-Positive Breast Cancer. J. Clin. Oncol. 24, 3726-3734.

5 Sparano J. A., Gray R. J., Makower D. F., Pritchard K. I., Albain K. S., Hayes D. F., Geyer C. E., Dees E. C., Goetz M. P., Olson J. A., Lively T., Badve S. S., Saphner T. J., Wagner L. I., Whelan T. J., Ellis M. J., Paik S., Wood W. C., Ravdin P. M. et al. (2018) Adjuvant Chemotherapy Guided by a 21-Gene Expression Assay in Breast Cancer. N. Engl. J. Med. 379, 111-121.

6 Van De Wetering M., Francies H. E., Francis J. M., Bounova G., Iorio F., Pronk A., Van Houdt W., Van Gorp J., Taylor-Weiner A., Kester L., McLaren-Douglas A., Blokker J., Jaksani S., Bartfeld S., Volckman R., Van Sluis P., Li V. S. W., Seepo S., Sekhar Pedamallu C. et al. (2015) Prospective derivation of a living organoid biobank of colorectal cancer patients. Cell. 161, 933-945.

7 Sachs N., de Ligt J., Kopper O., Gogola E., Bounova G., Weeber F., Balgobind A. V., Wind K., Gracanin A., Begthel H., Korving J., van Boxtel R., Duarte A. A., Lelieveld D., van Hoeck A., Ernst R. F., Blokzijl F., Nijman I. J., Hoogstraat M. et al. (2018) A Living Biobank of Breast Cancer Organoids Captures Disease Heterogeneity. Cell. 172, 373-386.e10.

8 Kopper O., de Witte C. J., Lõhmussaar K., Valle-Inclan J. E., Hami N., Kester L., Balgobind A. V., Korving J., Proost N., Begthel H., van Wijk L. M., Revilla S. A., Theeuwsen R., van de Ven M., van Roosmalen M. J., Ponsioen B., Ho V. W. H., Neel B. G., Bosse T. et al. (2019) An organoid platform for ovarian cancer captures intra- and interpatient heterogeneity. Nat. Med. 25, 838-849.

9 Ooft S. N., Weeber F., Dijkstra K. K., McLean C. M., Kaing S., van Werkhoven E., Schipper L., Hoes L., Vis D. J., van de Haar J., Prevoo W., Snaebjornsson P., van der Velden D., Klein M., Chalabi M., Boot H., van Leerdam M., Bloemendal H. J., Beerepoot L. V et al. (2019) Patient-derived organoids can predict response to chemotherapy in metastatic colorectal cancer patients. Sci. Transl. Med. 11, eaay2574.

10 Gabrin M., Brower S., McDonald S., Gallion H., Nanavati P., Dawn Rice S., Chattopadhyay A. Chemo-sensitivity assays using tumor cells exhibiting persistent phenotypic characteristics (2006) US20100143948A1 2006.

11 Romero-Calvo I., Weber C. R., Ray M., Brown M., Kirby K., Nandi R. K., Long T. M., Sparrow S. M., Ugolkov A., Qiang W., Zhang Y., Brunetti T., Kindler H., Segal J. P., Rzhetsky A., Mazar A. P., Buschmann M. M., Weichselbaum R., Roggin K. et al. (2019) Human Organoids Share Structural and Genetic Features with Primary Pancreatic Adenocarcinoma Tumors. Mol. Cancer Res. 17, 70-83.

12 Hoffman R. M. Methods to determine cancer treatment using robotic high-throughput drug sensitivity testing (2017) US20190011434A1 2017.

13 Larsson P.-A., Carlsson G., Gustaysson B., Graf W., Glimelius B. (1996) Different Intravenous Administration Techniques for 5-Fluorouracil Pharmacokinetics and Pharmacodynamic Effects. Acta Oncol. (Madr). 35, 207-212.

14 Ehrsson H., Wallin I., Yachnin J. (2002) Pharmacokinetics of Oxaliplatin in Humans. Med. Oncol. 19, 261-266.

15 Matsumoto H., Okumura H., Murakami H., Kubota H., Higashida M., Tsuruta A., Tohyama K., Hirai T. (2015) Fluctuation in Plasma 5-Fluorouracil Concentration During Continuous 5-Fluorouracil Infusion for Colorectal Cancer. Anticancer Res. 35, 6193-9.

16 Saam J., Critchfield G. C., Hamilton S. A., Roa B. B., Wenstrup R. J., Kaldate R. R. (2011) Body Surface Area-based Dosing of 5-Fluoruracil Results in Extensive Interindividual Variability in 5-Fluorouracil Exposure in Colorectal Cancer Patients on FOLFOX Regimens. Clin. Colorectal Cancer. 10, 203-206.

17 Michels B. E., Mosa M. H., Grebbin B. M., Yepes D., Darvishi T., Hausmann J., Urlaub H., Zeuzem S., Kvasnicka H. M., Oellerich T., Farin H. F. (2019) Human colon organoids reveal distinct physiologic and oncogenic Wnt responses. J. Exp. Med. 216, 704-720.

18 Liston D. R., Davis M. (2017) Clinically Relevant Concentrations of Anticancer Drugs: A Guide for Nonclinical Studies. Clin. Cancer Res. 23, 3489-3498.

19 Guo Y., Xiong B.-H., Zhang T., Cheng Y., Ma L. (2016) XELOX vs. FOLFOX in metastatic colorectal cancer: An updated meta-analysis. Cancer Invest. 34, 94-104.

20 Ducreux M., Bennouna J., Hebbar M., Ychou M., Lledo G., Conroy T., Adenis A., Faroux R., Rebischung C., Bergougnoux L., Kockler L., Douillard J.-Y. (2011) Capecitabine plus oxaliplatin (XELOX) versus 5-fluorouracil/leucovorin plus oxaliplatin (FOLFOX-6) as first-line treatment for metastatic colorectal cancer. Int. J. Cancer. 128, 682-690.

1. An in vitro method for evaluating the efficacy of a clinical drug treatment regimen utilizing tumor cells, comprising the steps of:
a. selecting a clinical drug treatment regimen to be evaluated comprising administrations of more than one drug and/or more than one administration of a drug, noting the sequence and timing of drug administrations included in said drug treatment regimen and identifying the one or more active substance(s) relevant for each noted administration;
b. selecting in vitro culture parameters corresponding to each noted drug administration comprising:
i. selecting concentration(s) for incubation with each identified active substance corresponding to the in vivo concentration of said active substance typical for said drug treatment regimen;
ii. selecting duration(s) of exposure for incubation with each identified active substance corresponding to the duration of clinically effective drug exposure to said active substance in said drug treatment regimen; and
iii. selecting time point(s) of addition for initiation of incubation with each identified active substance corresponding to the sequence and timing of drug administrations relevant for clinical exposure to said active substance in said drug treatment regimen;
wherein the combination of the selected time point(s), duration(s) of exposure and concentrations(s) imitates the clinical exposure profile typical for said drug treatment regimen;
c. providing a culture of tumor cells for an in vitro assay;
d. culturing the tumor cells in vitro with addition of each identified active substance(s) at the selected time point(s) of addition in accordance with the sequence and incubating in the presence of the selected concentration(s) of said active substance(s) for the selected duration(s);
e. determining the phenotypical changes of the tumor cells due to effect of the active substance(s); and
f. evaluating the efficacy of the drug treatment regimen based on the observed phenotypical changes.

2. The method according to claim 1, wherein:
a. the method comprises identification of the active substance(s) of said drug treatment regimen that are simultaneously present in the body based on the sequence and timing of drug administrations and pharmacokinetic parameters of said active substance(s); and
b. wherein the selected time point(s) of addition are for sequential initiation of incubation, based on the simultaneous presence in the body.

3. The method according to claim 1, wherein the method comprises selecting a total in vitro assay time Tₜₒₜ, and wherein for each identified active substance, the duration Tₑₓ of exposure for in vitro assay and in vitro concentration C_{in vitro} for assay, are selected to satisfy the following rules:
$$i . 0.01 \cdot {C}_{max in vivo} \leq {C}_{in vitro} \leq 3 \cdot {C}_{max in vitro} ;$$
$$ii . \frac{0.2 \cdot {AUC}_{in vivo}}{{C}_{in vitro}} \leq {T}_{ex} \leq \frac{3 \cdot {AUC}_{in vivo}}{{C}_{in vitro}} ;$$
where the in vivo maximal concentration C_{max in vivo} for each active substance and the in vivo area under concentration-time curve AUC_{in vivo} for the total in vitro assay time Tₜₒₜ for each active substance are based on clinical data for the selected regimen.

4. The method according to claim 1, wherein the selected sequence and time point(s) of addition match the sequence of administrations in the selected regimen within a margin of ±8 hours.

5. The method according to claim 1, wherein the drug treatment regimen is selected from the list consisting of FOLFOX, FOLFIRI, XELOX and capecitabine monotherapy.

6. The method according to claim 1, wherein any active substances which do not possess direct cytotoxic or cytostatic effect are ignored.

7. The method according to claim 1, wherein an active substance is the drug itself, an active drug metabolite or the drug or its metabolite in complex with other compound(s).

8. The method according to claim 1, wherein culturing the tumor cells is performed in 2D cell culture, suspension cell culture or 3D cell culture including tumor organoids or their combination with stromal and immune cells.

9. The method according to claim 1, wherein the cells are cell lines or primary cells.

10. The method according to claim 1, wherein the drug treatment regimen is a chemotherapy, targeted therapy, immunotherapy treatment regimen for cancer or their combinations.

11. The method according to claim 1, wherein the tumor cells are derived from an individual patient afflicted with a tumor disease, and the method is performed to evaluate the efficacy of a chemotherapy, targeted therapy, immunotherapy or their combinational drug treatment regimen in the treatment of said tumor disease.

12-19. (canceled)

20. The method according to claim 1, wherein the drug treatment regimen is considered effective when this regimen inhibits cell proliferation, stops cell proliferation and/or causes cell death.

21. The method according to claim 1, wherein the selected concentration(s) satisfy the conditions as defined in Table 1.

22. The method according to claim 1, wherein the selected duration(s) satisfy the conditions as defined in Table 1.

23. The method according to claim 1, wherein the selected time point(s) of addition satisfy the conditions as defined in Table 1.

24. (canceled)

25. A method for selecting in vitro culture parameters for a method according to claim 1, comprising the steps of:
a. selecting a drug treatment regimen to be evaluated;
b. selecting a total in vitro assay time Tₜₒₜ;
c. identifying the sequence [l, . . . , I] and time T_{a in vivo}ⁱ ([T_{a in vivo}¹, . . . , T_{a in vivo}^{l}])of drug administrations included in said drug treatment regimen to be evaluated during Tₜₒₜ;
d. identifying the active substances Sₖⁱ ([S_{1, . . . ,}ⁱ S_{K}ⁱ]) relevant for each identified administration i;
e. identifying clinical half life time T_{1/2[k]}ⁱ ([T_{1/2[1], . . . ,}ⁱ T_{1/2[K]}ⁱ]) for each active substance Sₖⁱ for each administration i;
f. identifying clinical time T_{max[k]}ⁱ([T_{max[1], . . . ,}ⁱ T_{max[K]}ⁱ]) for each active substance when concentration of this active substance reaches its maximum;
g. identifying clinical time of presence T_{pr[k]}ⁱ ([T_{pr[i], . . . ,}ⁱ T_{pr[K]}ⁱ]) for each active substance Sₖⁱ when this active substance is present in plasma in the range between T_{max[k]}ⁱ+0.1·T_{1/2[k]}ⁱ and T_{max[k]}ⁱ+10·T_{1/2[k]}ⁱ;
h. determining whether there is simultaneous presence of each identified active substance Sₖⁱ and any of the active substance(s) S_{y}^{x} of the preceding administration(s) and current administration i using the following rules:
i. if T_{a in vivo}ⁱ−T_{a in vivo}^{x}≥T_{pr[y]}^{x} then the substances are considered not to be present simultaneously in the body;
ii. if T_{a in vivo}ⁱ−T_{a in vivo}^{x}<T_{pr[y]}^{x} then the substances are considered to be present simultaneously in the body;
i. identifying the in vivo area under the curve AUC_{in vivo[k]}ⁱ for the period Tₜₒₜ for each active substance Sₖⁱ for each administration i, based on clinical data for the selected regimen;
j. identifying the in vivo maximal concentration C_{max in vivo[k]}ⁱ for each active substance Sₖⁱ for each administration i, based on clinical data for the selected regimen;
k. for each identified active substance Sₖⁱ, selecting at least one combination (when applicable: several combinations (1, . . . , N) of duration T_{ex[k][n]}ⁱ of exposure for in vitro assay and in vitro concentration C_{in vitro [k][n]}ⁱ for assay, to satisfy the following rules:
$$i . 0.01 \cdot {C}_{max in vivo [ k ]}^{i} \leq {C}_{in vitro [ k ] [ 1 ]}^{i} \leq 3 \cdot {C}_{max in vivo [ k ]}^{i} ;$$
$$ii . \frac{0.2 \cdot {AUC}_{in vivo [ k ]}^{i}}{{C}_{in vitro [ k ] [ 1 ]}^{i}} \leq {T}_{ex [ k ] [ 1 ]}^{i} \leq \frac{3 \cdot {AUC}_{in vivo [ k ]}^{i}}{{C}_{in vitro [ k ] [ 1 ]}^{i}} ;$$
l. optionally: for each identified active substance Sₖⁱ, if C_{in vitro[k][1]}ⁱ·T_{ex[k][1]}ⁱ<3·AUC_{in vivo[k]}ⁱ then additional combinations of duration T_{ex[k][n]}ⁱ of exposure for assay and concentration C_{in vitro [k ][n]}ⁱ for assay are identified to satisfy the following rules:
0.01·C_{max in vivo[k]}ⁱ≤C_{in vitro [k][n]}ⁱ≤3·C_{max in vivo[k]}ⁱ; i.
0.2·AUC_{in vivo[k]}ⁱ≤AUC_{in vitro[k]}ⁱ≤3·AUC_{in vivo[k]}ⁱ, where AUC_{in vitro[k]}ⁱ=AUC_{in vitro[k][1]}ⁱ+ . . . +AUC_{in vitro[k][N]}ⁱ and AUC_{in vitro[k][n]}ⁱ=C_{in vitro [k][n]}ⁱ·T_{ex[k][n]}ⁱ; ii.
m. for all identified administrations and all identified active substances sum of all durations of exposure for assay must satisfy the following criterion: ΣT_{ex[k][n]}ⁱ≤Tₜₒₜ;
n. for each identified active substance Sₖ¹ relevant for the first identified administration time point of addition in vitro T_{a in vitro[k]}¹ for assay must satisfy the following rule:
0≤T_{a in vitro[k]}¹≤T_{max[k]}¹; and
o. for each identified active substance Sₖⁱ relevant for any administration except the first one selecting time point of addition in vitro T_{a in vitro[k]}ⁱ for assay must satisfy the following rules:
i. if identified active substance Sₖⁱ and any of the active substance(s) S_{y}^{x} of the preceding administration(s) and current administration I are present simultaneously in the body then T_{a in vitro[y]}^{x}≤T_{a in vitro[k]}ⁱ≤T_{a in vitro[y]}^{x}+ΣₙT_{ex[y][n]}^{x};
ii. if identified active substance Sₖⁱ and any of the active substance(s) S_{y}^{x} of the preceding administration(s) and current administration I are not present simultaneously in the body then T_{a in vitro[y]}^{x}+ΣₙT_{ex[y][n]}^{x}≤T_{a in vitro[k]}ⁱ<Tₜₒₜ.

26. The method according to claim 25, wherein Tₜₒₜ is the time needed for the tumor cells to increase by at least 1.2-fold in cell numbers in culture.

27. The method according to claim 25, wherein Tₜₒₜ≤72 h.

28-31. (canceled)

32. A device adapted to perform the method according to claim 1, comprising:
a. a programmable control unit (7) for executing device functions to perform the method;
b. an incubator unit (8) for in vitro tumor cell culture;
c. a storage unit (4) for the active compound(s);
d. a liquid handling unit (2) for administering the active compound(s) to cultured tumor cells; and
e. a detector for determining the phenotypical changes of tumor cells (6) due to effect of the active substance(s).

33-39. (canceled)

40. A method treatment for cancer in a patient in need thereof, comprising performing an analysis in accordance with the method of claim 1 using cells derived from the patient, whereby various drug treatment regimens are tested using the cells, and based on the results obtained, said patient is subsequently administered with a drug treatment regimen deemed most likely to be beneficial for said patient to treat the cancer.
